(19) United States
(12) Patent Application Publication
(10) Pub. No.: US 2021/0284991 A1
(43) Pub. Date: Sep. 16, 2021

(54) Yeast Cell Extract Assisted Construction of DNA Molecules

(71) Applicant: Novozymes A/S, Bagsvaerd (DK)

(72) Inventor: Doreen Bohan, Fairfield, CA (US)

(73) Assignee: Novozymes A/S, Bagsvaerd (DK)

(21) Appl. No.: 16/462,859

(22) PCT Filed: Nov. 17, 2017
(86) PCT No.: PCT/US2017/062246

§ 371 Date: May 21, 2019

Related U.S. Application Data
(60) Provisional application No. 62/425,005, filed on Nov. 21, 2016.

(51) Int. Cl. C12N 15/10; C12Q 1/6813

(52) U.S. Cl. CPC C12N 15/1031; C12Q 1/6813; C12N 15/1027

(57) ABSTRACT

The present invention relates to a method for constructing DNA molecules, comprising: contacting a plurality of double-stranded DNA fragments with a cell-free extract of a yeast strain in a single in vitro reaction to combine the plurality of DNA fragments into the DNA molecules, wherein each of the DNA fragments has a 5′ end and a 3′ end, and wherein the DNA fragments combine with each other when the 5′ end of one fragment has at least 15 bp that are homologous with the 3′ end of another fragment.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to a method for constructing DNA molecules from DNA fragments with a yeast cell extract.

### Description of the Related Art

Traditional DNA cloning methods involve restriction cloning using restriction enzymes to generate appropriate DNA fragments, modifying the ends of the DNA fragments to generate blunt or sticky ends, and ligating the DNA fragments to generate plasmids or vectors. Such cloning requires the availability of suitable restriction enzymes and modifying enzymes, e.g., Klenow fragment, ligase.

In recent years, alternative methods for cloning have been developed involving in vitro assembly of DNA.

U.S. Pat. No. 8,609,374 discloses methods for assembling double-stranded DNA fragments into DNA molecules in a single in vitro recombination reaction by mixing the double-stranded DNA fragments with a bacterial extract derived from a RecA deficient bacterial strain to assemble the DNA fragments into DNA molecules.

Zhang et. al. (Nucleic Acids Research 40: e55, 2012) disclose a cloning method termed “Seamless Ligation Cloning Extract” (SLiCE) that utilizes bacterial cell extracts to assemble multiple DNA fragments into recombinant DNA molecules in a single in vitro recombination reaction.

Fisher et. al. (Front. Bioeng. Biotechnol. 1: 12, 2013) disclose an ex vivo DNA assembly method that uses cellular lysates derived from Escherichia coli for joining double-stranded DNA with short end homologies embedded within primers.

However, there is a need in the art for new methods for constructing DNA molecules from DNA fragments, which are inexpensive, rapid, and reliable.

The present invention relates to a method for constructing DNA molecules from DNA fragments with a cell-free extract of a yeast strain.

### SUMMARY OF THE INVENTION

The present invention relates to a method for constructing DNA molecules, said method comprising: contacting a plurality of double-stranded DNA fragments with a cell-free extract of a yeast strain in a single in vitro reaction to combine the plurality of DNA fragments into the DNA molecules, wherein each of the DNA fragments has a 5′ end and a 3′ end, and wherein the DNA fragments combine with each other when the 5′ end of one fragment has at least 15 bp that are homologous with the 3′ end of another fragment.

In one aspect, the method further comprises transforming the resulting DNA molecules into a host cell and isolating single colony transformants comprising each of the DNA molecules. In another aspect, the method further comprises recovering the DNA molecules from the single colony transformants. In another aspect, the method further comprises transforming a recovered DNA molecule encoding a polypeptide having a biological activity of interest into a recombinant host cell, wherein the DNA molecule is operably linked to one or more control sequences that direct the production of the polypeptide. In another aspect, the method further comprises cultivating the recombinant host cells under conditions suitable for producing the polypeptide having a biological activity of interest. In another aspect, the method further comprises recovering the polypeptide having a biological activity of interest.

### BRIEF DESCRIPTION OF THE FIGURE

FIG. 1A, FIG. 1B, FIG. 1C, and FIG. 1D are pictorial representations of different cloning capabilities where the black blocks indicate homologous sequence(s) at insertion site(s) and the wavy grey lines indicate non-homologous sequence removal during cloning. FIG. 1A shows an illustration of standard cloning and multi-fragment cloning. FIG. 1B shows an illustration of non-homologous sequence removal directly downstream of a homologous insertion site. FIG. 1C shows an illustration of non-homologous sequence removal directly upstream of a homologous insertion site. FIG. 1D shows an illustration of non-homologous sequence removal both upstream and downstream of a homologous insertion site.

### DEFINITIONS

cDNA: The term “cDNA” means a DNA molecule that can be prepared by reverse transcription from a mature, spliced, mRNA molecule obtained from a eukaryotic or prokaryotic cell. cDNA lacks intron sequences that can be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon, such as ATG, GTG, or TTG, and ends with a stop codon, such as TAA, TAG, or TGA. The coding sequence can be a genomic DNA, cDNA, synthetic DNA, or a combination thereof.

Control sequences: The term “control sequences” means nucleic acid sequences necessary for expression of a polynucleotide encoding a polypeptide of interest. Each control sequence can be native (i.e., from the same gene) or heterologous or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences can be provided with linkers for the purpose of introducing specific restriction sites facilitating ligation of the control sequences with the coding region of the polynucleotide encoding a polypeptide.

DNA library: The term “DNA library” means a collection of recombinant expression vectors or plasmids containing inserts (DNA fragments) from a single genome, two or more genomes, mutated DNA, shuffled DNA, and the like. The vectors can be linear or closed circular plasmids. The origin of the insert DNA can be genomic, cDNA, semisynthetic, synthetic, or any combinations thereof.

DNA ligase: The term “DNA ligase” means an enzyme that seals nicks in DNA molecules by forming a phosphodiester bond between adjacent nucleotides which have free 5′-phosphate and 3′-hydroxyl groups.

DNA polymerase: The term “DNA polymerase” means an enzyme that synthesizes DNA by copying a template strand. DNA polymerase synthesizes DNA in the 5′ to 3′ direction by successively adding nucleotides to the free 3′ hydroxyl group of the growing strand. The template strand determines the order of addition of nucleotides via Watson-Crick base pairing.

Downstream: The term “downstream” means the direction in which a nucleic acid is synthesized, i.e., on the 3′ side of any given site in a DNA or RNA.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Host cell: The term “host cell” means any cell type that is susceptible to transformation, transfection, transduction, or the like with a nucleic acid construct or expression vector comprising a polynucleotide. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Klenow fragment: The term “Klenow fragment, also known as “Klenow enzyme”, means the larger of two fragments of E. coli DNA polymerase I formed after cleavage with a protease such as subtilisin. The larger fragment retains 5′→3′ polymerase and 3′→5′ exonuclease activities.

Mutant: The term “mutant” means a mutated polynucleotide encoding a polypeptide variant comprising one or more alterations, such as substitutions, insertions, deletions, and/or truncations of one or more specific amino acid residues at one or more specific positions of a parent polypeptide.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Parent polypeptide: The term “parent polypeptide” means a polypeptide to which one or more alterations, e.g., substitutions, insertions, deletions, and/or truncations, are made to produce polypeptide variants. The parent can be a naturally occurring (wild-type) polypeptide, or the parent protein can be a variant of a naturally occurring polypeptide that has been modified or altered in the amino acid sequence, prepared by any suitable means. A parent may also be an allelic variant of a polypeptide that is encoded by any of two or more alternative forms of a gene occupying the same chromosomal locus.

Recombination: The term “recombination” means a process wherein nucleic acids associate with each other in regions of homology, leading to interstrand DNA exchange between those sequences. “Homologous recombination” is defined herein as recombination in which no changes in the nucleotide sequences occurs within the regions of homology relative to the input nucleotide sequences. Recombination may also occur by non-homologous recombination. “Non-homologous recombination” is defined herein as recombination where any mode of DNA repair incorporating strand exchange results in a nucleotide sequence different from any of the recombining sequences.

Restriction enzyme: The term “restriction enzyme” means an endonuclease that digests DNA at or near specific recognition nucleotide sequences known as restriction sites. Restriction enzymes are also referred to as “restriction endonucleases”. Naturally occurring restriction endonucleases are categorized into four groups (Types I, II III, and IV) based on their composition and enzyme cofactor requirements, the nature of their target sequence, and the position of their DNA cleavage site relative to the target sequence. Type I enzymes cleave at sites remote from a recognition site and require both ATP and S-adenosyl-L-methionine to function. Type II enzymes cleave within or at short specific distances from a recognition site and most require magnesium. Type II enzymes are commonly used in traditional cloning. Type III enzymes cleave at sites a short distance from a recognition site and require ATP. Type IV enzymes target modified DNA, e.g., methylated, hydroxymethylated, and glucosyl-hydroxymethylated DNA.

Transformant: The term “transformant” means a microorganism genetically modified by introduction of a polynucleotide fragment. The process by which the DNA is introduced, e.g., transformation, transfection, transduction, and the like, to produce a transformant can entail DNA derived from a different organism than the host, or can entail use of DNA that is derived from the same species or the host cell.

Upstream: The term “upstream” means the opposite direction in which a nucleic acid is synthesized, i.e., on the 5′ side of any given site in a DNA or RNA.

Variant: The term “variant” means a polypeptide having biological activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position.

Wild-type polypeptide: The term “wild-type polypeptide” denotes a polypeptide expressed by a naturally occurring microorganism.

Reference to “about” a value or parameter herein includes aspects that are directed to that value or parameter per se. For example, description referring to “about X” includes the aspect “X”.

As used herein and in the appended claims, the singular forms “a,” “or,” and “the” include plural referents unless the context clearly dictates otherwise. It is understood that the aspects of the invention described herein include “consisting” and/or “consisting essentially of” aspects.

Unless defined otherwise or clearly indicated by context, all technical and scientific terms used herein have the same meaning as commonly understood by one of ordinary skill in the art to which this invention belongs.

### DETAILED DESCRIPTION OF THE INVENTION

The present invention relates to a method for constructing DNA molecules, said method comprising: contacting a plurality of double-stranded DNA fragments with a cell-free extract of a yeast strain in a single in vitro reaction to combine the plurality of DNA fragments into the DNA molecules, wherein each of the DNA fragments has a 5′ end and a 3′ end, and wherein the DNA fragments combine with each other when the 5′ end of one fragment has at least 15 bp that are homologous with the 3′ end of another fragment.

In one aspect, the method further comprises transforming the resulting DNA molecules into a host cell and isolating single colony transformants comprising each of the DNA molecules. In another aspect, the method further comprises recovering the DNA molecules from the single colony transformants. In another aspect, the method further comprises transforming a recovered DNA molecule encoding a polypeptide having a biological activity of interest into a recombinant host cell, wherein the DNA molecule is operably linked to one or more control sequences that direct the production of the polypeptide. In another aspect, the method further comprises cultivating the recombinant host cells under conditions suitable for producing the polypeptide having a biological activity of interest. In another aspect, the method further comprises recovering the polypeptide having a biological activity of interest.

The method of the present invention is based on in vitro recombination between regions of homology in double-stranded DNA fragments using a cell-free extract of a yeast strain. The method allows for efficient restriction site-independent cloning of DNA fragments into linearized vectors.

There are several advantages associated with the method of the present invention. First, there is no need to digest a gene fragment with the same restriction enzyme to clone a gene fragment into a vector. Second, the method does not require the addition of exogenous enzymes to combine the plurality of DNA fragments into the DNA molecules. Such enzymes include DNA ligase, DNA polymerase, and Klenow fragment (having polymerase activity and 3′→5′ exonuclease activity). Third, the method does not require removal of non-homologous sequence directly downstream, upstream, or downstream and upstream of the homologous insertion site. Fourth, the method allows a rapid way of combining multiple fragments at once. For example, at least 5 fragments of DNA can be combined in one reaction.

### Cell-Free Yeast Extracts

The cell-free yeast extract can be obtained from any Saccharomyces strain. In one aspect, the Saccharomyces strain is Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis. In a preferred aspect, the Saccharomyces strain is Saccharomyces cerevisiae. In another preferred aspect, the Saccharomyces strain is Saccharomyces carlsbergensis. In another preferred aspect, the Saccharomyces strain is Saccharomyces diastaticus. In another preferred aspect, the Saccharomyces strain is Saccharomyces douglasii. In another preferred aspect, the Saccharomyces strain is Saccharomyces kluyveri. In another preferred aspect, the Saccharomyces strain is Saccharomyces norbensis. In another preferred aspect, the Saccharomyces strain is Saccharomyces oviformis. In a more preferred aspect, the Saccharomyces strain is Saccharomyces cerevisiae.

A cell-free extract of the Saccharomyces strain can be prepared using standard methods known in the art. For example, the Saccharomyces strain is cultivated in a suitable medium and then the cells are recovered by centrifugation. The recovered cells are then subjected to a suitable lysis procedure, e.g., mechanical disruption, in a buffer to produce an extract. An example of a useful buffer is 20 mM Bis-Tris, 1 mM dithiothreitol, 50 mM KCl, and 10% glycerol, pH 6.5. Cells and cellular debris can then be removed by, for example, centrifugation and/or filtration, to produce the cell-free extract. The cell-free extract can then be stored as a 50% glycerol solution at −80° C. for at least a year.

The protein concentration of the extract can be any protein concentration suitable for practicing the methods of the present invention. In one aspect, the protein concentration is preferably about 0.1 to about 50 μg of protein per μl of extract, more preferably about 1 to about 25 μg of protein per μl of extract, even more preferably about 1 to about 10 μg of protein per μl of extract, and most preferably about 1 to about 5 μg of protein per μl of extract. The protein concentration can be determined by the bicinchoninic add (BCA) protein assay or any other suitable protein assay.

### DNA Fragments

The origin of the DNA fragments can be genomic DNA, cDNA, semisynthetic DNA, synthetic DNA, or any combinations thereof. The DNA fragments can be obtained from a single genome, two or more genomes, mutated DNA, shuffled DNA, and the like. The DNA fragments can also be a linearized expression vector and a polynucleotide comprising a gene encoding a polypeptide having a biological activity of interest.

The DNA fragments have a 5′ end and a 3′ end wherein the 5′ end has a phosphate group at the 5′ carbon of the sugar and the 3′ end has a free hydroxyl group at the 3′ carbon of the sugar. Two nucleotides on opposite complementary DNA strands that are connected via hydrogen bonds are known as a base pair (bp), i.e., guanine (G) forms a base pair with cytosine (C) and adenine (A) forms a base pair with thymine (T).

The DNA fragments can combine with each other when the 5′ end of one fragment has at least 15 bp, at least 20 bp, at least 30 bp, at least 40 bp, at least 50 bp, at least 100 bp, or at least 200 bp that are homologous with the 3′ end of another fragment. Sequences of base pairs are “homologous” if the sequences are identical. The homologous regions should contain a sufficient number of nucleotides, such as at least 15 bp, which are highly homologous with the corresponding nucleotide sequence to enhance the probability of homologous recombination.

Each of the DNA fragments can be of any suitable size in the methods of the present invention. In one aspect, the DNA fragments range in size from about 15 bp to about 20 kb. In another aspect, the DNA fragments are at least 15 bp, at least 100 bp, at least 500 bp, at least 1000 bp, at least 5000 bp, at least 10,000 bp, at least 15,000 bp, or at least 20,000 bp in size.

The DNA fragments can also comprise non-homologous sequences of up to at least 1,000 bp adjacent to the homologous end of the fragment. Sequences of base pairs are “non-homologous” if the sequences are not identical.

Multiple DNA fragments can be combined with each other in the methods of the present invention. For example, 2, 3, 4, 5, 6, 7, 8, 9, 10 fragments can be combined in one reaction.

The DNA fragments can be prepared or obtained by a number of methods known in the art. The DNA fragments can be prepared by PCR amplification of nucleic acid including DNA, cDNA, and RNA, and can be isolated using standard methods known in the art. For example, cDNA probes can be obtained from the total polyadenylated mRNA isolated from cells of an organism or organisms using standard methods and reverse transcribed into total cDNA. The DNA fragments can also be prepared by digesting a double-stranded DNA with one or more restriction enzymes. The DNA fragments can also be prepared synthetically by established standard methods, e.g., the phosphoamidite method described by Beaucage and Caruthers, 1981, Tetrahedron Letters 22: 1859-1869, or the method described by Matthes et al., (1984), EMBO Journal 3: 801-805.

The DNA fragments can be obtained from any organism, including, but not limited to, microorganisms, plants, and mammals.

In a preferred aspect, the DNA fragments are obtained from a bacterium such as a Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

In a more preferred aspect, the DNA fragments are obtained from Bacillus alkalophilus, Bacillus amyloliquefaciens, Bacillus brevis, Bacillus circulans, Bacillus coagulans, Bacillus lautus, Bacillus lentus, Bacillus licheniformis, Bacillus megaterium, Bacillus stearothermophilus, Bacillus subtilis, Bacillus thuringiensis, Streptomyces lividans, or Streptomyces murinus.

In another preferred aspect, the DNA fragments are obtained from a yeast such as a Candida, Kluyveromyces, Pichia, Saccharomyces, Schizosaccharomyces, or Yarrowia strain; or a filamentous fungus such as an Acremonium, Aspergillus, Aureobasidium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, or Trichoderma strain.

In a more preferred aspect, the DNA fragments are obtained from Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis.

In another more preferred aspect, the DNA fragments are obtained from Aspergillus aculeatus, Aspergillus awamori, Aspergillus fumigatus, Aspergillus foetidus, Aspergillus japonicus, Aspergillus nidulans, Aspergillus niger, Aspergillus oryzae, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Talaromyces emersonii, Talaromyces leycettanus, Trichoderma harzianum, Trichoderma koningii, Trichoderma longibrachiatum, Trichoderma reesei, or Trichoderma viride.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

The DNA fragments can also be obtained from a DNA material purified directly from or amplified from an environmental sample, including a collection of uncultivated organisms, such as from a soil sample, a freshwater sample, a saltwater sample, an insect gut, an animal stomach, waste water, sludge, or sediment. (Liles et al., 2003, Appl. Environ. Microbiol. 69: 2684-2691; Beja et al., 2000, Science 289: 1902-1906; Tyson et al., 2004, Nature 428: 37-43; Venter et al., 2004, Science 304: 66-74, U.S. Pat. No. 6,723,504).

In the methods of the present invention, the DNA fragments can be combined with a linearized vector to produce DNA libraries. The DNA libraries are a collection of recombinant vectors containing DNA fragments (also known as inserts). As mentioned above, the DNA fragments can be from a single genome, two or more genomes, mutated DNA, shuffled DNA, and the like. The DNA fragments can be genomic DNA fragments, cDNA fragments, semisynthetic DNA fragments, synthetic DNA fragments, or any combinations thereof.

The vectors used in a DNA library can be any plasmid that can be subjected to recombinant DNA procedures to introduce the inserts according to the methods of the present invention. The plasmid can be a shuttle plasmid which can be maintained and replicated in E. coli and which can then be used to transform a host cell for expression.

The vector preferably contains one or more selectable markers that permit easy selection of transformed cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosylaminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, pyrG, and tk genes.

The selectable marker may be a dual selectable marker system as described in WO 2010/039889. In one aspect, the dual selectable marker is a hph-tk dual selectable marker system.

In one aspect, each DNA fragment or insert comprises a nucleotide sequence encoding a polypeptide having biological activity of interest or a fragment thereof which retains biological activity.

The polypeptide can be any polypeptide having a biological activity of interest. The polypeptide can be native or heterologous to the host cell employed. Moreover, the polypeptide can be a variant of a parent polypeptide. The term “polypeptide” is not meant herein to refer to a specific length of the encoded product and, therefore, encompasses peptides, oligopeptides, and proteins. The term “polypeptide” also encompasses naturally occurring allelic or engineered variants of a polypeptide.

In a preferred aspect, the polypeptide is an antibody, antigen, antimicrobial peptide, enzyme, growth factor, hormone, immunodilator, neurotransmitter, receptor, reporter protein, structural protein, transcription factor, and transporter.

In a more preferred aspect, the polypeptide is an oxidoreductase, transferase, hydrolase, lyase, isomerase, or ligase. In a most preferred aspect, the polypeptide is an alpha-glucosidase, aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, glucocerebrosidase, alpha-glucosidase, beta-glucosidase, invertase, laccase, lipase, lytic polysaccharide monooxygenase, mannosidase, mutanase, oxidase, pectinolytic enzyme, peroxidase, phospholipase, phytase, polyphenoloxidase, proteolytic enzyme, ribonuclease, transglutaminase, urokinase, or xylanase.

The DNA fragments can comprise a plurality of mutated genes encoding variant polypeptides. Such variants can comprise a modification of a parent polypeptide such as a substitution, insertion and/or deletion at one or more positions of the parent polypeptide.

Mutations can be introduced by procedures known in the art, such as PCR or error prone PCR. The PCR amplification can be combined with a mutagenesis step using a suitable physical or chemical mutagenizing agent, e.g., one which induces transitions, transversions, inversions, scrambling, substitutions, deletions, and/or insertions. In a preferred aspect of the present invention, the DNA fragments are prepared under conditions resulting in a low, medium or high random mutagenesis frequency. To obtain low mutagenesis frequency the nucleotide sequence(s) (comprising the DNA fragment(s)) can be prepared by a standard PCR amplification method (U.S. Pat. No. 4,683,202 or Saiki et al., 1988, Science 239: 487-491). A medium or high mutagenesis frequency can be obtained by performing the PCR amplification under conditions which reduce the fidelity of replication by a thermostable polymerase and increase the misincorporation of nucleotides, for instance as described by Deshler, 1992, GATA 9: 103-106; Leung et al., 1989, BioTechniques 1: 11-15.

Other methods for producing mutated genes are known in the art, such as oligonucleotide-directed mutagenesis, assembly PCR, in vivo mutagenesis, site-specific mutagenesis, region-directed mutagenesis, and oligonucleotide cassette mutagenesis (Reidhaar-Olson and Sauer, 1988, Science 241: 53-57; Bowie and Sauer, 1989, Proc. Natl. Acad. Sci. USA 86: 2152-2156; WO 95/17413; WO 95/22625; Lowman et al., 1991, Biochem. 30: 10832-10837; U.S. Pat. No. 5,223,409; WO 92/06204; Derbyshire et al., 1986, Gene 46: 145; Ner et al., 1988, DNA 7: 127).

### In Vitro Reaction

In the methods of the present invention, a plurality of double-stranded DNA fragments are contacted with a cell-free extract of a yeast strain in a single in vitro reaction to combine the plurality of DNA fragments into the DNA molecules. The DNA fragments combine with each other when the 5′ end of one fragment has at least 15 bp that are homologous with the 3′ end of another fragment.

The cell-free yeast extract is preferably present in a saturating amount when the cell-free extract is contacted with the plurality of double-stranded DNA fragments. For example, the amount of the cell-free extract based on protein concentration can be at least 2-fold, at least 5-fold, at least 10-fold, at least 25-fold, at least 50-fold, or at least 100-fold greater than the amount of the DNA fragment(s). Lower and higher amounts can also be used.

The reaction can be performed at any suitable temperature, pH, and period of time. For example, the reaction can be performed at a temperature in the range of 20° C. to 45° C., e.g., 30° C. or 37° C., and a pH in the range of 4.0 to 8.0, e.g., pH 5.0 or 6.5, for a period of time of minutes to hours, e.g., 1 minute to 5 hours, such as 1 hour.

### Transformation

The methods of the present invention further comprise transforming the resulting DNA molecules into a host cell and isolating single colony transformants comprising each of the DNA molecules.

In the methods of the present invention, any suitable host cell can be used to isolate single colony transformants. The host cell can be a prokaryote (for example, Gram-positive bacteria such as Bacillus, Clostridium, Enterococcus, Geobacillus, Lactobacillus, Lactococcus, Oceanobacillus, Staphylococcus, Streptococcus, Streptomyces, or Gram-negative bacteria such as Campylobacter, E. coli, Flavobacterium, Fusobacterium, Helicobacter, Ilyobacter, Neisseria, Pseudomonas, Salmonella, and Ureaplasma), or a eukaryote (for example, yeast such as Candida, Hansenula, Kluyveromyces, Pichia, Saccharomyces, Schizosaccharomyces, or Yarrowia, or filamentous fungi such as Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma).

The Bacillus host cell can be, but is not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis cells.

The yeast host cell can be, but is not limited to, a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The filamentous fungal host cell can be, but is not limited to, an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

In one aspect, an E. coli strain is preferred especially for DNA libraries. Any E. coli strain can be utilized to isolate single colony transformants of the DNA molecules, e.g., a DNA library introduced into the E. coli strain. Examples of E. coli strains useful in the practice of the present invention include, but are not limited to, DH5α™ (Invitrogen) [F-80d/acZΔM15 Δ(lacZYA-argF) U169 recA1 endA1 hsdR17(rₖ⁻, mₖ+) phoA supE44 λ⁻ thi-1 gyrA96 relA1]; ONE SHOT® INVaIphaF′ (Invitrogen) [F′ endA1 recA1 hsdR17 (rk−, mk+) supE44 thi-1 gyrA96 relA1 φ80lacZ.M15.(lacZYA-argF)U169 λ−]; Top10 (Invitrogen) F⁻ mcrA Δ(mrr-hsdRMS-mcrBC) ϕ80lacZΔM15 ΔlacX74 recA1 araΔ139 Δ(ara-leu)7697 galU galK rpsL (Str^{R}) endA1 nupG; and E. coli STELLAR™ competent cells (Clontech Laboratories, Inc.).

The introduction of the DNA molecules into E. coli can be achieved through the use of chemically competent or electroporation competent E. coli cells. For example, introduction of a DNA library can be accomplished with SURE® Electroporation-Competent Cells (Stratagene, U.S. Pat. Nos. 6,338,965, 6,040,184, 6,017,748, and 5,552,314 and equivalent foreign patents), XL1-Blue Electroporation-Competent Cells (Stratagene, La Jolla, Calif., U.S. Pat. Nos. 6,338,965 and 6,040,184), XL10-GOLD® Ultracompetent Cells (Stratagene, La Jolla, Calif., U.S. Pat. Nos. 5,512,468 and 5,707,841), and SURE® Competent Cells (Stratagene, La Jolla, Calif., U.S. Pat. Nos. 6,017,748, 5,707,841, 5,552,314, and 5,512,468; U.S. Pat. Nos. 6,017,748, 5,552,314; 6,338,965, 6,040,184, 6,017,748, and 5,552,314).

Transformants are selected using a selective medium, e.g., a liquid or solid nutrient-containing mixture, which allows growth of transformed microorganisms, but does not allow growth of untransformed cells. Selection can be obtained by inclusion of a substance in the medium that is toxic to the untransformed organism, such that when an expression vector containing a selective marker, as described herein, is present in a transformed microorganism, expression of the marker detoxifies the toxin or otherwise provides resistance to it. Selection can also be obtained by utilizing a microorganism that is unable to grow in the absence of a supplemental nutrient. For example, an auxotrophic mutant can be subjected to transformation and selection can be provided by screening in the absence of a needed metabolite that can only be synthesized by strains containing an expression vector which provides a polynucleotide sequence required for synthesis of the metabolite.

The isolation of single colony E. coli transformants of the DNA molecules can be accomplished manually or using a colony-picking device. Any commercially available device can be used. Such devices include, but are not limited to, a Genetix QPIX™ (Genetix Limited) and a VERSARRAY™ Colony Picker and Arrayer System (Bio-Rad Laboratories, Inc.).

The single colony E. coli transformants can be transferred into individual wells of a multiwell plate, which can be accomplished manually or using a colony-picking device, as described above.

### Recovery of DNA Molecules

In another aspect of the methods of the present invention, the methods further comprise recovering the DNA molecules from the single colony transformants.

In the methods of the present invention, recovering a DNA molecule from each of the single colony transformants, e.g., E. coli transformants, can be performed in any format known in the art. The recovery of the DNA is preferably performed in the individual wells of a multiwell plate. However, preparation of the DNA can be accomplished using any procedure known in the art. See, for example, Sambrook, et al., 1989, Molecular Cloning: a Laboratory Manual Cold Spring Harbor Laboratory Press, 2^{nd} Ed., pp 1.21-1.49, for methods for boiling, SDS, and alkali lysis, and purification by methods such as cesium chloride gradient. DNA can be isolated using a 96-well Miniprep Kit protocol of Advanced Genetic Technologies Corporation as modified by Utterback et al. (1995, Genome Sci. Technol. 1: 1-8). In addition, rolling-circle amplification can be used to prepare DNA from single colony E. coli transformants (Nelson et al., 2002, Biotechniques, June, Suppl: 44-47).

In a preferred aspect, recovering the DNA molecules is automated. For example, plasmid DNA from E. coli strains can be prepared using a robotic device, e.g., a BIOROBOT® Universal System (QIAGEN Inc.). Plasmid DNA can also be isolated from the cultures using a Qiabot Miniprep Station (QIAGEN Inc.) following the manufacturer's instructions. Another device is the AutoGenprep 960 instrument (AutoGen, Inc.) that is a fully automated high-throughput instrument for DNA extraction in a 96-well format.

In another preferred aspect, the DNA molecules recovered from the single colony transformants are transformed into host cells to express the DNA molecules.

### Expression of DNA Molecules

The DNA molecules recovered from the single colony transformants can be transformed into host cells to express the DNA molecules. Consequently, the method of the present invention further comprises transforming a recovered DNA molecule encoding a polypeptide having a biological activity of interest into a recombinant host cell, wherein the DNA molecule is operably linked to one or more control sequences that direct the production of the polypeptide.

A recovered DNA molecule encoding a polypeptide having a biological activity of interest can be manipulated in a variety of ways to provide for expression of the polypeptide. Manipulation of the DNA molecule prior to its insertion into a vector can be desirable or necessary depending on the expression vector. The techniques for modifying polynucleotides utilizing recombinant DNA methods are well known in the art.

The DNA molecule can be operably linked to one or more control sequences that direct the expression of the coding sequence of the polypeptide in a suitable host cell under conditions compatible with the control sequences.

The control sequence can be a promoter, a polynucleotide that is recognized by a host cell for expression of a DNA molecule encoding a polypeptide. The promoter contains transcriptional control sequences that mediate the expression of the polypeptide. The promoter can be any polynucleotide that shows transcriptional activity in the host cell including mutant, truncated, and hybrid promoters, and can be obtained from genes encoding extracellular or intracellular polypeptides either homologous or heterologous to the host cell.

Examples of suitable promoters for directing transcription of the nucleic acid constructs in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Dania (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the DNA molecule encoding the polypeptide. Any terminator that is functional in the host cell can be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the DNA molecule encoding the polypeptide. Any leader that is functional in the host cell can be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the DNA molecule and, when transcribed, is recognized by the host cell as a signal to add polyadenosine residues to transcribed mRNA. Any polyadenylation sequence that is functional in the host cell can be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the DNA molecule may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence can be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell can be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence can be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the DNA molecule encoding the polypeptide would be operably linked to the regulatory sequence.

The DNA molecule can be inserted into a recombinant expression vector for expression in a host cell of choice. The recombinant expression vector can be any vector (e.g., a plasmid or virus) that can be conveniently subjected to recombinant DNA procedures and can bring about expression of the DNA molecule. The choice of the vector will typically depend on the compatibility of the vector with the host cell into which the vector is to be introduced. The vector can be a linear or closed circular plasmid.

The vector can be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector can be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, can be used.

The vector preferably contains one or more selectable markers, as described herein, that permit easy selection of transformed, transfected, transduced, or the like cells. A selectable marker is a gene the product of which provides for biocide or viral resistance, resistance to heavy metals, prototrophy to auxotrophs, and the like.

The vector preferably contains an element(s) that permits integration of the vector into the host cell's genome or autonomous replication of the vector in the cell independent of the genome.

For integration into the host cell genome, the vector may rely on the DNA molecule's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements can be any sequence that is homologous with the target sequence in the genome of the host cell. Furthermore, the integrational elements can be non-encoding or encoding polynucleotides. On the other hand, the vector can be integrated into the genome of the host cell by non-homologous recombination.

For autonomous replication, the vector may further comprise an origin of replication enabling the vector to replicate autonomously in the host cell in question. The origin of replication can be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a DNA molecule can be inserted into a host cell to increase production of a polypeptide. An increase in the copy number of the DNA molecule can be obtained by integrating at least one additional copy of the sequence into the host cell genome or by including an amplifiable selectable marker gene with the DNA molecule where cells containing amplified copies of the selectable marker gene, and thereby additional copies of the DNA molecule, can be selected for by cultivating the cells in the presence of the appropriate selectable agent.

In the methods of the present invention, each of the DNA molecules can be introduced into separate suspensions of a host cell, e.g., protoplasts of a filamentous fungus, to obtain transformants thereof, using any format known in the art. Each transformant will contain one or more copies of an individual DNA molecule. Transformation is preferably performed in the individual wells of a multiwell plate.

The host cell can be any cell useful in the recombinant production of a polypeptide, e.g., a prokaryote or a eukaryote.

The prokaryotic host cell can be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell can be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The introduction of DNA into a Bacillus cell can be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell can be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell can be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell can be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell can be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell can be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell can be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell can be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell can be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism can be fermentative.

The filamentous fungal host cell can be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell can be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells can be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast can be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

The host cells are cultivated in a nutrient medium suitable for production of the polypeptide using methods known in the art. For example, the cells can be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes μlace in a suitable nutrient medium comprising carbon and nitrogen sources and inorganic salts, using procedures known in the art. Suitable media are available from commercial suppliers or can be prepared according to published compositions (e.g., in catalogues of the American Type Culture Collection).

In another aspect, the method further comprises recovering the polypeptide having a biological activity of interest. If the polypeptide is secreted into the nutrient medium, the polypeptide can be recovered directly from the medium. If the polypeptide is not secreted, it can be recovered from cell lysates.

The polypeptide can be detected by measuring activity or a property using methods known in the art that are specific for the polypeptide of interest. These detection methods include, but are not limited to, use of specific antibodies, formation of an enzyme product, or disappearance of an enzyme substrate. Properties include, but are not limited to, altered temperature-dependent activity profile, thermostability, pH activity, pH stability, substrate specificity, product specificity, and chemical stability. The measurement of expression of the polypeptide or a property of the polypeptide can be accomplished manually or by automation.

Measurement of activity or a property can be automated. Any device that allows for automation can be used, e.g., robotic devices. Commercially available devices include, but are not limited to, a BIOMEK® Fx liquid handling robot (Beckman Coulter, Inc.), ORCA® plate handling robotic arm (Beckman Coulter, Inc.), SciClone ALH300 Work Station (Caliper Life Sciences), and QBot robot (Genetix Limited). In order to increase the number of individual activity assays performed in a given time, the activity is conveniently assayed in a high-throughput screening system using multiwell plates. Multiwell plates include, but are not limited to, 96-well HARD-SHELL® microplates, (MJ Research), COSTAR® 3370 96-well clear polystyrene plate (Corning), Polypropylene Ultra Rigid Deep-Well Plate (ABgene), COSTAR® 3950 1536-well assay plates (Corning), COSTAR® 3706 384-well clear bottom polystyrene plates (Corning), and COSTAR® 24-well cell culture cluster (Corning). Such screening techniques are well known in the art, see, e.g., Taylor et al., 2002, J. Biomolec. Screening 7: 554-569; Decker et al., 2003, Appl. Biochem. Biotech. 105-108: 689-703; Dove, 1999, Nature Biotech. 17: 859-863, and Kell, 1999, Trends in Biotechnology 17: 89-91.

The polypeptide can be recovered using methods known in the art. For example, the polypeptide can be recovered from the nutrient medium by conventional procedures including, but not limited to, collection, centrifugation, filtration, extraction, spray-drying, evaporation, or precipitation. In one aspect, a whole fermentation broth comprising the polypeptide is recovered.

The polypeptide can be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

The present invention is further described by the following examples that should not be construed as limiting the scope of the invention.

### EXAMPLES

### Media and Solutions

2XYT/ampicillin plates were composed of 16 g of tryptone, 10 g of yeast extract, 5 g of NaCl, 15 g of Bacto agar, 1 ml of ampicillin at 100 mg/ml, and water to 1 liter.

SOC medium was composed of 20 g of tryptone, 5 g of yeast extract, 0.5 g of NaCl, 10 ml of 250 mM KCl, and water to 1 liter.

TE buffer was composed of 10 ml of 1 M Tris pH8.0, 2 ml of 0.5 M EDTA pH 8.0, and water to 1 liter.

TBE was composed of 10.8 g of Tris Base, 5.5 g of boric acid, 4 ml of 0.5 M EDTA pH 8.0, and water to 1 liter.

Yeast extract buffer was composed of 20 mM Bis-Tris, 1 mM dithiothreitol, 50 mM KCl, and 10% glycerol, pH 6.5.

YPG medium was composed of 10 g of Bacto yeast extract, 20 g of Bacto peptone, 2% glucose, and water to 1 liter.

YPD plates were composed of 10 g of Bacto yeast extract, 20 g of Bacto peptone, 20 g of Bacto agar, 40 ml of 50% glucose, and water to 1 liter.

### Example 1: Isolation and Quantification of Yeast Extracts

Saccharomyces cerevisiae strain JG169 (U.S. Published Application 2009/0317866) and Saccharomyces cerevisiae strain HiP19 (WO 2014/072481) were used as sources to produce yeast extracts. The following protocol was used to produce yeast extract from each strain. The strain was streaked onto an YPD plate and incubated at 30° C. for 48-72 hours. A loop of yeast cells was collected from the plate and inoculated into a 500 ml glass shake flask containing 100 ml of YPG medium. The shake flask was incubated at 30° C. overnight at 250 rpm. The yeast culture was then transferred to a centrifuge bottle and centrifuged at 3,090×g for 10 minutes. The supernatant was decanted and the cell pellet was resuspended in 200 ml of water. The cells were then centrifuged again at 3090×g for 10 minutes. The supernatant was decanted and the cell pellet was transferred into a pre-chilled pestle containing 1-5 ml of liquid nitrogen. The frozen pellet was crushed slightly with a mortar and transferred to a pre-chilled SPEX® SamplePrep Freezer Mill vial containing a slug (Fisher Scientific) to pulverize the pellet. The vial was inserted into a SPEX® SamplePrep 6970EFM Freezer/Mill (Fisher Scientific) with the following settings: 6 cycles, precool 1 minute, run 2 minutes, cool 2 minutes, rate 12 cps. The pulverized pellet was transferred to a 50 ml conical tube, resuspended in 3-5 ml of yeast extract buffer, and centrifuged at 21,130×g for 5 minutes to remove cell debris. Following centrifugation, the supernatant was collected and an equal volume of 100% glycerol was added. Each yeast extract was then stored in EPPENDORF® LoBind microfuge tubes (Eppendorf) at −80° C.

A bicinchoninic acid (BCA) protein assay was used to quantify the yeast extracts. Prior to quantification the glycerol was removed from the sample. To remove the glycerol, 100 μl of prepared yeast extract was loaded onto a VIVASPIN™ 500 sample concentrator (GE Healthcare) and centrifuged at 21,130×g for at least 60 minutes. The column was washed twice with 100 μl of TE buffer and centrifuged at 21,130×g for at least 15 minutes. The protein was then eluted with 100 μl of TE buffer.

The BCA protein assay was performed using a BCA Protein Assay Kit (Thermo Fisher Scientific). Bovine serum albumin (BSA) standards were set at 0 μg, 5 μg, 10 μg, 25 μg and 50 μg to generate a standard curve. The yeast extract was loaded either with no dilution or dilutions in water at 75%, 50%, 25%, 10%, and 5%. BCA Reagent A was mixed with BCA Reagent B at 9.8 ml and 200 μl, respectively. A volume of 25 μl of either the standards or the samples was added to 200 μl of the mixed BCA reagent in a CORNING® COSTAR® 96 well flat bottom culture plate (Sigma-Aldrich) and incubated at room temperature for 30 minutes. The plate was read on a SPECTRAMAX® 340PC Microplate Reader (Molecular Devices) with the wavelength set to 562 nm. The yeast extract samples were then quantified against a best fit trend generated by the BSA control standards. The yeast extract of Saccharomyces cerevisiae strain JG169 contained 2.64 μg of protein per μl. The yeast extract of Saccharomyces cerevisiae strain HiP19 contained 1.77 μg of protein per μl.

### Example 2: In Vitro DNA Assembly and Transformation Using Yeast Extract

The assembly method first involves the generation of a linearized vector and insert fragment(s) containing homologous sequences to the desired insertion region or to the adjacent fragment in the case of multiple fragment assembly; second, an in vitro reaction using yeast extract to assemble all DNA segments; and third, transformation into E. coli competent cells.

In vitro DNA assembly was performed with a mixture composed of at least 100 ng of linearized vector, at least 300 ng of each insert(s), 2.64 μg (2 μl) of yeast extract (Example 1), 1 μl of yeast extract buffer, and water to 10 μl. The DNA assembly mixture was incubated at 37° C. for 1 hour. One μl of the DNA assembly mixture was transformed into 50 μl of E. coli STELLAR™ competent cells (Clontech Laboratories, Inc.). The transformation reaction was incubated on ice for 30 minutes, heat shocked at 42° C. for 45 seconds, and incubated on ice for an additional 2 minutes. A 100 μl volume of SOC medium was added and the transformed cells were spread onto agar plates containing appropriate antibiotics, e.g., 2XYT/ampicillin plates.

### Example 3: Construction of Plasmid pSPT001

pSPT001 plasmids were constructed by piecing either the entirety or 2 to 4 segmented portions of a codon optimized coding sequence encoding an Aspergillus aculeatus beta-glucosidase (SEQ ID NO: 1 for the DNA sequence and SEQ ID NO: 2 for the deduced amino acid sequence) into expression vector pBM120 (U.S. Pat. No. 8,586,330).

Primer pairs (Table 1) were designed to amplify by PCR either the whole or segments of the A. aculeatus beta-glucosidase codon optimized coding sequence from plasmid pDFng181-25. Fifty picomoles of each of the primer pairs were used in a PCR composed of 10 ng of plasmid pDFng181-15, 1× EXPAND® High Fidelity PCR buffer with MgCl₂ (Roche Diagnostics Corporation), 0.25 mM each of dATP, dTTP, dGTP, and dCTP, and 2.6 units of EXPAND® Enzyme Mix (Roche Diagnostics Corporation) in a final volume of 50 μl. The PCR with 20 to 30 bp overhangs was performed using a thermal cycler programmed for 1 cycle at 94° C. for 2 minutes; 30 cycles each at 94° C. for 20 seconds, 60° C. for 30 seconds, and 72° C. for 3 minutes; and a final elongation at 72° C. for 7 minutes. The PCR with 40 to 50 bp overhangs was performed using a thermal cycler programmed for 1 cycle at 94° C. for 2 minutes; 20 cycles each at 94° C. for 20 seconds, 72° C. for 3 minutes; and a final elongation at 72° C. for 7 minutes. The heat block then went to a 10° C. soak cycle.

TABLE 1
Primer description for generation of
fragments used to generate pSPT001 plasmids

| Primer | | |
|---|---|---|
| # | Primer name | Primer sequences (5′→3′) |
| 1 | SPT_AaBG F | CTCTATATACACAACTGGCCATGAAGCTCAGTT |
| | | GGCTCGA (SEQ ID NO: 3) |
| 2 | SPT_AaBG R | GTGTCAGTCACCTCTAGTTACTATTGGACCTTG |
| | | GGCAGAG (SEQ ID NO: 4) |
| 3 | SPT_AaBG F30 | TTCCTCAATCCTCTATATACACAACTGGCCATG |
| | | AAGCTCAGTTGGCTCGA (SEQ ID NO: 5) |
| 4 | SPT_AaBG R30 | CTACCGCCAGGTGTCAGTCACCTCTAGTTACTA |
| | | TTGGACCTTGGGCAGAG (SEQ ID NO: 6) |
| 5 | SPT_AaBG F40 | CTTCTCTTCCTTCCTCAATCCTCTATATACACA |
| | | ACTGGCCATGAAGCTCAGTTGGCTCGA |
| | | (SEQ ID NO: 7) |
| 6 | SPT_AaBG R40 | GATTGATTGTCTACCGCCAGGTGTCAGTCACCT |
| | | CTAGTTACTATTGGACCTTGGGCAGAG |
| | | (SEQ ID NO: 8) |
| 7 | SPT_AaBG R1_2 | GCAGCCGTTAGCACCGTACGAGTTGGAACCAGC |
| | | GTCTTCACCGAGGATAG (SEQ ID NO: 9) |
| 8 | SPT_AaBG F2_2 | GGTTCCAACTCGTACGGTGCTAACGGCTGCTCT |
| | | GACCGCGGCTGTGATAA (SEQ ID NO: 10) |
| 9 | SPT_AaBG R1_3 | GGGCAGAGCCGACACCGCTGTGGTGAGCACCCC |
| | | AGTCACTCATGACGAAG (SEQ ID NO: 11) |
| 10 | SPT_AaBG F2_3 | GTGCTCACCACAGCGGTGTCGGCTCTGCCCTCG |
| | | CTGGGTTGGATATGTCG (SEQ ID NO: 12) |
| 11 | SPT_AaBG R2_3 | ACTCTCCTGACCGGGCAAGCCGGCCCAGAGGAT |
| | | GGCAGTGACGTTGGGGT (SEQ ID NO: 13) |
| 12 | SPT_AaBG F3_3 | CTCTGGGCCGGCTTGCCCGGTCAGGAGAGTGGC |
| | | AACTCCCTGGCTGACGT (SEQ ID NO: 14) |
| 13 | SPT_AaBG R1_4 | CACGTTGGAGCTGATCGTGTCGGAGATGTTGAA |
| | | ACCATATCCGGCGGCCT (SEQ ID NO: 15) |
| 14 | SPT_AaBG F2_4 | AACATCTCCGACACGATCAGCTCCAACGTGGAT |
| | | GACAAGACCATTCACGA (SEQ ID NO: 16) |
| 15 | SPT_AaBG R3_4 | CATGGCCAAACTCATAAATGGGGGTCTCATTGC |
| | | GCTTGTCAAATCCACGG (SEQ ID NO: 17) |
| 16 | SPT_AaBG F4_4 | ATGAGACCCCCATTTATGAGTTTGGCCATGGCT |
| | | TGAGCTACACCACCTTT (SEQ ID NO: 18) |
| 17 | SPT_AaBG R1_2 40 | TCAGAGCAGCCGTTAGCACCGTACGAGTTGGAA |
| | | CCAGCGTCTTCACCGAGGATAGCCACT |
| | | (SEQ ID NO: 19) |
| 18 | SPT_AaBG F2_2 40 | ACGCTGGTTCCAACTCGTACGGTGCTAACGGCT |
| | | GCTCTGACCGCGGCTGTGATAACGGCA |
| | | (SEQ ID NO: 20) |
| 19 | SPT_AaBG R1_3 40 | AGCGAGGGCAGAGCCGACACCGCTGTGGTGAGC |
| | | ACCCCAGTCACTCATGACGAAGCCTTG |
| | | (SEQ ID NO: 21) |
| 20 | SPT_AaBG F2_3 40 | CTGGGGTGCTCACCACAGCGGTGTCGGCTCTGC |
| | | CCTCGCTGGGTTGGATATGTCGATGCC |
| | | (SEQ ID NO: 22) |
| 21 | SPT_AaBG R2_3 40 | TTGCCACTCTCCTGACCGGGCAAGCCGGCCCAG |
| | | AGGATGGCAGTGACGTTGGGGTGATCA |
| | | (SEQ ID NO: 23) |
| 22 | SPT_AaBG F3_3 40 | CCATCCTCTGGGCCGGCTTGCCCGGTCAGGAGA |
| | | GTGGCAACTCCCTGGCTGACGTGCTCT |
| | | (SEQ ID NO: 24) |
| 23 | SPT_AaBG R1_4 40 | TCATCCACGTTGGAGCTGATCGTGTCGGAGATG |
| | | TTGAAACCATATCCGGCGGCCTCTGCA |
| | | (SEQ ID NO: 25) |
| 24 | SPT_AaBG F2_4 40 | GTTTCAACATCTCCGACACGATCAGCTCCAACG |
| | | TGGATGACAAGACCATTCACGAGATGT |
| | | (SEQ ID NO: 26) |
| 25 | SPT_AaBG R3_4 40 | CAAGCCATGGCCAAACTCATAAATGGGGGTCTC |
| | | ATTGCGCTTGTCAAATCCACGGTAGTC |
| | | (SEQ ID NO: 27) |
| 26 | SPT_AaBG F4_4 40 | GCGCAATGAGACCCCCATTTATGAGTTTGGCCA |
| | | TGGCTTGAGCTACACCACCTTTAACTA |
| | | (SEQ ID NO: 28) |
| 27 | SPT_AaBG F50 | TCTATCCACACTTCTCTTCCTTCCTCAATCCTC |
| | | TATATACACAACTGGCCATGAAGCTCAGTTGGC |
| | | TCGA (SEQ ID NO: 29) |
| 28 | SPT_AaBG R50 | TAGCGAAATGGATTGATTGTCTACCGCCAGGTG |
| | | TCAGTCACCTCTAGTTACTATTGGACCTTGGGC |
| | | AGAG (SEQ ID NO: 30) |
| 29 | SPT_AaBG R1_2 50 | CGCGGTCAGAGCAGCCGTTAGCACCGTACGAGT |
| | | TGGAACCAGCGTCTTCACCGAGGATAGCCACTT |
| | | TTCG (SEQ ID NO: 31) |
| 30 | SPT_AaBG F2_2 50 | TGAAGACGCTGGTTCCAACTCGTACGGTGCTAA |
| | | CGGCTGCTCTGACCGCGGCTGTGATAACGGCAC |
| | | TCTT (SEQ ID NO: 32) |
| 31 | SPT_AaBG R1_3 50 | AACCCAGCGAGGGCAGAGCCGACACCGCTGTGG |
| | | TGAGCACCCCAGTCACTCATGACGAAGCCTTGG |
| | | AAGC (SEQ ID NO: 33) |
| 32 | SPT_AaBG F2_3 50 | AGTGACTGGGGTGCTCACCACAGCGGTGTCGGC |
| | | TCTGCCCTCGCTGGGTTGGATATGTCGATGCCT |
| | | GGAG (SEQ ID NO: 34) |
| 33 | SPT_AaBG R2_3 50 | GGGAGTTGCCACTCTCCTGACCGGGCAAGCCGG |
| | | CCCAGAGGATGGCAGTGACGTTGGGGTGATCAT |
| | | ACCA (SEQ ID NO: 35) |
| 34 | SPT_AaBG F3_3 50 | CACTGCCATCCTCTGGGCCGGCTTGCCCGGTCA |
| | | GGAGAGTGGCAACTCCCTGGCTGACGTGCTCTA |
| | | TGGC (SEQ ID NO: 36) |
| 35 | SPT_AaBG R1_4 50 | TCTTGTCATCCACGTTGGAGCTGATCGTGTCGG |
| | | AGATGTTGAAACCATATCCGGCGGCCTCTGCAA |
| | | CCTG (SEQ ID NO: 37) |
| 36 | SPT_AaBG F2_4 50 | ATATGGTTTCAACATCTCCGACACGATCAGCTC |
| | | CAACGTGGATGACAAGACCATTCACGAGATGTA |
| | | CCTT (SEQ ID NO: 38) |
| 37 | SPT_AaBG R3_4 50 | TAGCTCAAGCCATGGCCAAACTCATAAATGGGG |
| | | GTCTCATTGCGCTTGTCAAATCCACGGTAGTCA |
| | | ATGA (SEQ ID NO: 39) |
| 38 | SPT_AaBG F4_4 50 | GACAAGCGCAATGAGACCCCCATTTATGAGTTT |
| | | GGCCATGGCTTGAGCTACACCACCTTTAACTAC |
| | | TCTG (SEQ ID NO: 40) |

Bold letters represent coding sequence. The remaining sequence is homologous to vector sequence of pBM120.

Table 2 provides information for the construction of the pSPT001 plasmids including homologous overhang lengths, the number of fragment(s) made, the size of the fragments, and the primers used. The reaction products were isolated by 0.7% agarose gel electrophoresis using TBE buffer where a fragment band of correct size (Table 2) was observed. The PCRs were digested with Dpn I and purified using a NUCLEOSPIN® Gel and PCR Clean-up Kit (Clontech Laboratories, Inc.). One volume of sample was mixed with 2 volumes of Buffer NTI (Clontech Laboratories, Inc.). The mixture was loaded into a NUCLEOSPIN® Gel and PCR Clean-up Kit column and centrifuged for 30 seconds at 11,000×g. Flow-through was discarded. The column was washed with 700 μl of Buffer NT3 (Clontech Laboratories, Inc.) and centrifuged at 11,000×g for 30 seconds. Flow-through was discarded and the column was centrifuged at 11,000×g for an additional minute. The DNA was eluted by applying 30 μl of Buffer NE (Clontech Laboratories, Inc.) to the column for 1 minute and centrifuged at 11,000×g for 1 minute.

TABLE 2
Description of fragment generation for construction of pSPT001

| | | Forward | Reverse | Size of | |
|---|---|---|---|---|---|
| Overhang length | # of Gene segments | primer | primer | fragment | |
| 20 bp overhangs | Entire gene | 1 | 2 | 2.6 | kb |
| 30 bp overhangs | Entire gene | 3 | 4 | 2.6 | kb |
| | 2 equal size | 3 | 7 | 1.3 | kb |
| | segments | 8 | 4 | 1.3 | kb |
| | 3 equal size | 3 | 9 | 860 | bp |
| | segments | 10 | 11 | 860 | bp |
| | | 12 | 4 | 860 | bp |
| | 4 equal size | 3 | 13 | 645 | bp |
| | segments | 14 | 7 | 645 | bp |
| | | 8 | 15 | 645 | bp |
| | | 16 | 4 | 645 | bp |
| | 2 varying size | 3 | 19 | 860 | bp |
| | segments | 10 | 4 | 1.7 | kb |
| | 2 varying size | 3 | 13 | 645 | bp |
| | segments | 14 | 4 | 1.9 | kb |
| 40 bp overhangs | Entire gene | 5 | 6 | 2.6 | kb |
| | 2 equal size | 5 | 17 | 1.3 | kb |
| | segments | 18 | 6 | 1.3 | kb |
| | 3 equal size | 5 | 19 | 860 | bp |
| | segments | 20 | 21 | 860 | bp |
| | | 22 | 6 | 860 | bp |
| | 4 equal size | 5 | 23 | 645 | bp |
| | segments | 24 | 17 | 645 | bp |
| | | 18 | 25 | 645 | bp |
| | | 26 | 6 | 645 | bp |
| 50 bp overhangs | Entire gene | 27 | 28 | 2.6 | kb |
| | 2 equal size | 27 | 29 | 1.3 | kb |
| | segments | 30 | 28 | 1.3 | kb |
| | 3 equal size | 27 | 31 | 860 | bp |
| | segments | 32 | 33 | 860 | bp |
| | | 34 | 28 | 860 | bp |
| | 4 equal size | 27 | 35 | 645 | bp |
| | segments | 36 | 29 | 645 | bp |
| | | 30 | 37 | 645 | bp |
| | | 38 | 28 | 645 | bp |

Plasmid pBM120 was digested with Nco I and Pac I, isolated by 0.7% agarose gel electrophoresis using TBE buffer, and purified using a NUCLEOSPIN® TriPrep Kit (Macherey-Nagel). The gene fragment(s) and the digested vector were assembled using the yeast extract DNA assembly method described in Example 2 with the S. cerevisiae strain JG169 extract. One μl of the reaction was transformed into E. coli STELLAR™ competent cells. The transformation reaction was incubated on ice for 30 minutes, heat shocked at 42° C. for 45 seconds, and incubated on ice for an additional 2 minutes. A 100 μl volume of SOC medium was added and the transformed cells were spread onto 2XYT/ampicillin plates. Plasmid DNA was prepared from the E. coli transformants using a BIOROBOT® Universal System (QIAGEN Inc.). Transformants were confirmed by Bam HI and Nco I digestion and/or DNA sequencing. DNA sequencing of the insertion sites was performed with a Perkin-Elmer Applied Biosystems Model 377 XL Automated DNA Sequencer (Perkin-Elmer/Applied Biosystems, Inc.) using dye-terminator chemistry (Giesecke et al., 1992, Journal of Virology Methods 38: 47-60). The following primers (Table 3) were used for sequencing:

TABLE 3
Sequencing Primers

| | Primer | |
|---|---|---|
| Construct | name | Primer sequence (5′→3′) |
| pSPT001 | 996271 | ACTCAATTTACCTCTATCCACAC |
| | | TT (SEQ ID NO: 41) |
| | pAllo2R | CACATGACTTGGCTTCC |
| | | (SEQ ID NO: 42) |
| | AaBGseq1F | GTACTTTTCGCCGAAACTAT |
| | | (SEQ ID NO: 43) |
| | AaBGseq2F | ACGGCTTCAAGTATTTCTAC |
| | | (SEQ ID NO: 44) |
| | AaBGseq3F | TGGGCCGGCTTGCCCGGTCA |
| | | (SEQ ID NO: 45) |

### Example 4: Construction of pSPT002 Plasmids

pSPT002 plasmids were constructed by piecing either the entirety or 2 to 4 segmented portions of a codon optimized coding sequence encoding a Talaromyces leycettanus cellobiohydrolase I (CBHI; SEQ ID NO: 46 for the DNA sequence and SEQ ID NO: 47 for the deduced amino acid sequence) into expression vector pAllo2 (U.S. Pat. No. 7,393,664).

Primer pairs (Table 4) were designed to amplify by PCR either the whole or segments of the T. leycettanus CBHI codon optimized gene from plasmid pLsBf96. Fifty picomoles of each of the primers below were used in a PCR composed of 10 ng of plasmid pLsBf96, 1× EXPAND® High Fidelity PCR buffer with MgCl₂, 0.25 mM each of dATP, dTTP, dGTP, and dCTP, and 2.6 units of EXPAND® Enzyme Mix in a final volume of 50 μl. The PCR with the 20 to 30 bp overhangs was performed using a thermal cycler programmed for 1 cycle at 94° C. for 2 minutes; 30 cycles each at 94° C. for 20 seconds, 60° C. for 30 seconds, and 72° C. for 3 minutes; and a final elongation at 72° C. for 7 minutes. The heat block then went to a 10° C. soak cycle.

TABLE 4
Primer description for generation of
fragments used to generate pSPT002 plasmids

| Primer | Primer | |
|---|---|---|
| # | name | Primer sequences (5′→3′) |
| 39 | SPT_CBH1 | ATATACACAACTGGATTTACATGC |
| | 20F | TTTTGCAAGCCTTCCT |
| | | (SEQ ID NO: 48) |
| 40 | SPT_CBH1 | GTGTCAGTCACCTCTAGTTATTAC |
| | 20R | AGGCACTGGTAGTAGT |
| | | (SEQ ID NO: 49) |
| 41 | SPT_CBH1 | TCAATCCTCTATATACACAACTGG |
| | 30F | ATTTACATGCTTTTGCAAGCCTTC |
| | | CT (SEQ ID NO: 50) |
| 42 | SPT_CBH1 | CTACCGCCAGGTGTCAGTCACCTC |
| | 30R | TAGTTATTACAGGCACTGGTAGTA |
| | | GT (SEQ ID NO: 51) |
| 43 | SPT_CBH1 | TCTTCCTTCCTCAATCCTCTATAT |
| | 40F | ACACAACTGGATTTACATGCTTTT |
| | | GCAAGCCTTCCT |
| | | (SEQ ID NO: 52) |
| 44 | SPT_CBH1 | GATTGATTGTCTACCGCCAGGTGT |
| | 40R | CAGTCACCTCTAGTTATTACAGGC |
| | | ACTGGTAGTAGT |
| | | (SEQ ID NO: 53) |
| 45 | SPT_CBH1 | GTAGTGCTGTAAGTGCCACCACAG |
| | R1_2 | TTGTTGCCGGTGCACATGACCTGT |
| | | CC (SEQ ID NO: 54) |
| 46 | SPT_CBH1 | CAACAACTGTGGTGGCACTTACAG |
| | F2_2 | CACTACTCGCTATGCTGGCACTTG |
| | | CG (SEQ ID NO: 55) |
| 47 | SPT_CBH1 | TTGTTGGTTGGGTACTTGGCCATG |
| | R1_3 | CCGCCGTCGGCATCCATGGCAACC |
| | | AG (SEQ ID NO: 56) |
| 48 | SPT_CBH1 | CGGCGGCATGGCCAAGTACCCAAC |
| | F2_3 | CAACAAGGCTGGTGCGAAGTACGG |
| | | AA (SEQ ID NO: 57) |
| 49 | SPT_CBH1 | TGCAGAACTCGGTGGTGATGGAGT |
| | R2_3 | TGCCGGAGACGCCGCTGATCGTGG |
| | | AC (SEQ ID NO: 58) |
| 50 | SPT_CBH1 | CCGGCAACTCCATCACCACCGAGT |
| | F3_3 | TCTGCACGGCCCAGAAGCAGGCTT |
| | | TC (SEQ ID NO: 59) |
| 51 | SPT_CBH1 | TGGTAGGTTGTGTCATTCTCCATC |
| | R1_4 | AGATAGAGACGAGAGCCGACGTTC |
| | | TT (SEQ ID NO: 60) |
| 52 | SPT_CBH1 | CTATCTGATGGAGAATGACACAAC |
| | F2_4 | CTACCAGATCTTCAAGTTGCTGAA |
| | | CC (SEQ ID NO: 61) |
| 53 | SPT_CBH1 | GCATGTTGGCGGCGTGGTCGTCCC |
| | R3_4 | ACAAGCTCATGACGAGAACCATAC |
| | | CC (SEQ ID NO: 62) |
| 54 | SPT_CBH1 | GCTTGTGGGACGACCACGCCGCCA |
| | F4_4 | ACATGCTCTGGCTTGACAGCACCT |
| | | AC (SEQ ID NO: 63) |

Bold letters represent coding sequence. The remaining sequence is homologous to the insertion sites of pAllo2.

Table 5 provides information for the construction of the pSPT002 plasmids including homologous overhang lengths, the number of fragment(s) made, the size of the fragments, and the primers used. The reaction products were isolated by 0.7% agarose gel electrophoresis using TBE buffer where a fragment band of correct size (Table 5) was observed. The PCR mixture was digested with Dpn I and purified using a NUCLEOSPIN® TriPrep Kit.

TABLE 5
Description of fragment generation
for construction of plasmid pSPT002

| Overhang | | Forward | Reverse | Size of fragment | |
|---|---|---|---|---|---|
| length | # of gene segments | primer | primer | (−overhangs) | |
| 20 bp | Entire gene | 39 | 40 | 1.6 | kb |
| overhangs | | | | | |
| 30 bp | Entire gene | 41 | 42 | 1.6 | kb |
| overhangs | 2 equal size segments | 41 | 45 | 790 | bp |
| | | 46 | 42 | 790 | bp |
| | 3 equal size segments | 41 | 47 | 527 | bp |
| | | 48 | 49 | 527 | bp |
| | | 50 | 42 | 527 | bp |
| | 4 equal size segments | 41 | 51 | 395 | bp |
| | | 52 | 45 | 395 | bp |
| | | 46 | 53 | 395 | bp |
| | | 54 | 42 | 395 | bp |
| 40 bp | Entire gene | 43 | 44 | 1.6 | kb |
| overhangs | | | | | |

Plasmid pAllo2 was digested with Nco I and Pac I, isolated by 0.7% agarose gel electrophoresis using TBE buffer, and purified using a NUCLEOSPIN® TriPrep Kit. The gene fragment(s) and the digested vector were assembled using the yeast extract DNA assembly method described in Example 2 with the S. cerevisiae strain JG169 extract. One μl of the reaction was transformed into E. coli STELLAR™ competent cells according to Example 3. Plasmid DNA was prepared from the E. coli transformants using a BIOROBOT® Universal System. Transformants were confirmed by Bam HI and Nco I digestion and/or DNA sequencing. DNA sequencing of the insertion sites was performed with a Perkin-Elmer Applied Biosystems Model 377 XL Automated DNA Sequencer using dye-terminator chemistry (Giesecke et al., 1992, supra). The primers shown in Table 6 were used for sequencing.

TABLE 6
Sequencing Primers

| | Primer | |
|---|---|---|
| Construct | name | Primer sequence (5′→3′) |
| pSPT002 | pAllo2F | TGTCCCTTGTCGATGCG |
| | | (SEQ ID NO: 64) |
| | pAllo2R | CACATGACTTGGCTTCC |
| | | (SEQ ID NO; 65) |

### Example 5: Construction of Plasmid pSPT003

Plasmid pSPT003 was constructed by cloning the entire gene of a Gliomastix murorum alpha-L-arabinofuranosidase (SEQ ID NO: 66 for the DNA sequence and SEQ ID NO: 67 for the deduced amino acid sequence) into expression vector pMJ09 (U.S. Pat. No. 8,318,458).

Primer pairs (shown below) were designed to amplify by PCR the G. murorum α-L-arabinofuranosidase coding sequence from plasmid pDFng194-5. Fifty picomoles of each of the primers were used in a PCR composed of 10 ng of plasmid pDFng194-5, 1× EXPAND® High Fidelity PCR buffer with MgCl₂, 0.25 mM each of dATP, dTTP, dGTP, and dCTP, and 2.6 units of EXPAND® Enzyme Mix in a final volume of 50 μl. The PCR with the 20 to 30 bp overhangs was performed using a thermal cycler programmed for 1 cycle at 94° C. for 2 minutes; 30 cycles each at 94° C. for 20 seconds, 60° C. for 30 seconds, and 72° C. for 1.5 minutes; and a final elongation at 72° C. for 7 minutes. The heat block then went to a 10° C. soak cycle.

SPT_GH62 F20:
(SEQ ID NO: 68)
5′-GTCAACCGCGGACTGCGCACATGAAGTTCCATCTGGCATC-3′
SPT_GH62 R20:
(SEQ ID NO: 69)
5′-GGCTTTCGCCACGGAGCTTACTAAGTGCACTGAGAGTACC-3′

Bold letters represent coding sequence. The remaining sequence is homologous to the insertion sites in pMJ09.

The reaction products were isolated by 0.7% agarose gel electrophoresis using TBE buffer where a 1.2 kb product band was observed. The PCR mixture was digested with Dpn I and purified using a NUCLEOSPIN® TriPrep Kit.

Plasmid pMJ09 was digested with Nco I and Pac I, isolated by 0.7% agarose gel electrophoresis using TBE buffer, and purified using a NUCLEOSPIN® TriPrep Kit. The gene fragment and the digested vector were assembled using the yeast extract DNA assembly method described in Example 2 with the S. cerevisiae strain JG169 extract. One μl of the reaction was transformed into E. coli STELLAR™ competent cells according to Example 3. Plasmid DNA was prepared from the E. coli transformants using a BIOROBOT® Universal System. Transformants were confirmed by Sal I digestion and/or DNA sequencing. DNA sequencing of the insertion sites was performed with a Perkin-Elmer Applied Biosystems Model 377 XL Automated DNA Sequencer using dye-terminator chemistry (Giesecke et al., 1992, supra). The primers shown in Table 7 were used for sequencing.

TABLE 7
Sequencing Primers

| Construct | Primer name | Primer sequence (5′→3′) |
|---|---|---|
| pSPT003 | pMJ09Fwd | CCCATCTACTCATCAACTCA |
| | | (SEQ ID NO: 70) |
| | pMJ09Rev | ATAAATCACCCGGGGCCATG |
| | | (SEQ ID NO: 71) |

### Example 6: Construction of Plasmid pSPT004

Plasmid pSPT004 was constructed by cloning a codon optimized coding sequence encoding an Aspergillus aculeatus beta-glucosidase (SEQ ID NO: 1 for DNA sequence and SEQ ID NO: 2 for the deduced amino acid sequence) into expression vector pBM120 while removing 196 bp of unwanted non-homologous sequence directly downstream of the insertion site in the pBM120 vector.

Primer pairs (shown below) were designed to amplify by PCR the Aspergillus aculeatus beta-glucosidase codon optimized gene from plasmid pDFng181-25 (Example 3). Fifty picomoles of each of the primers above were used in a PCR composed of 10 ng of plasmid pDFng181-15, 1× EXPAND® High Fidelity PCR buffer with MgCl₂, 0.25 mM each of dATP, dTTP, dGTP, and dCTP, and 2.6 units of EXPAND® Enzyme Mix in a final volume of 50 μl. The PCR with the 20 to 30 bp overhangs was performed using a thermal cycler programmed for 1 cycle at 94° C. for 2 minutes; 30 cycles each at 94° C. for 20 seconds, 60° C. for 30 seconds, and 72° C. for 3 minutes; and a final elongation at 72° C. for 7 minutes. The heat block then went to a 10° C. soak cycle.

SPT_AaBG F40:
(SEQ ID NO: 72)
5′-CTTCTCTTCCTTCCTCAATCCTCTATATACACAACTGGCCATG
AAGCTCAGTTGGCTCGA-3′
SPT_AaBG-term R40:
(SEQ ID NO: 73)
5′-CAGGGACTGTCTGTCTGGTCTTCTACACGAAGGAAAGAGCCTA
TTGGACCTTGGGCAGAG-3′

Bold letters represent coding sequence. The remaining sequence is homologous to the region surrounding the insertion sites of pBM120.

The reaction products were isolated by 0.7% agarose gel electrophoresis using TBE buffer where a 2.66 kb product band was observed. The PCR mixture was digested with Dpn I and purified using a NUCLEOSPIN® TriPrep Kit.

Plasmid pBM120 was digested with Nco I and Pac I, isolated by 0.7% agarose gel electrophoresis using TBE buffer, and purified using a NUCLEOSPIN® TriPrep Kit. The gene fragment and the digested vector were assembled using the yeast extract DNA assembly method described in Example 2 with the S. cerevisiae strain JG169 extract. One μl of the reaction was transformed into E. coli STELLAR™ competent cells according to Example 3. Plasmid DNA was prepared from the E. coli transformants using a BIOROBOT® Universal System. Transformants were confirmed by Bam HI and Nco I digestion and/or DNA sequencing. DNA sequencing of the insertion sites was performed with a Perkin-Elmer Applied Biosystems Model 377 XL Automated DNA Sequencer using dye-terminator chemistry (Giesecke et al., 1992, supra). The primers shown in Table 8 were used for sequencing.

TABLE 8
Sequencing Primers

| Construct | Primer name | Primer sequence (5′→3′) |
|---|---|---|
| pSPT004 | pAllo2R | CACATGACTTGGCTTCC |
| | | (SEQ ID NO: 74) |
| | AaBGseq3F | TGGGCCGGCTTGCCCGGTCA |
| | | (SEQ ID NO: 75) |
| | AfBGseq4F | CATTTATGAGTTTGGCCATG |
| | | (SEQ ID NO: 76) |

### Example 7: Construction of Plasmid pSPT005

Plasmid pSPT005 was constructed by cloning a codon optimized coding sequence encoding an Aspergillus aculeatus beta-glucosidase (SEQ ID NO: 1 for the DNA sequence and SEQ ID NO: 2 for the deduced amino acid sequence) into expression vector pBM120 while removing 614 bp of unwanted non-homologous sequence directly upstream of the insertion site in the pBM120 vector.

The primer pair (shown below) was designed to amplify by PCR the Aspergillus aculeatus beta-glucosidase codon optimized gene from plasmid pDFng181-25 (Example 3). Fifty picomoles of each of the primers above were used in a PCR composed of 10 ng of plasmid pDFng181-15, 1× EXPAND™ High Fidelity PCR buffer with MgCl₂, 0.25 mM each of dATP, dTTP, dGTP, and dCTP, and 2.6 units of EXPAND™ Enzyme Mix in a final volume of 50 μl. The PCR with the 20 to 30 bp overhangs was performed using a thermal cycler programmed for 1 cycle at 94° C. for 2 minutes; 30 cycles each at 94° C. for 20 seconds, 60° C. for 30 seconds, and 72° C. for 3 minutes; and a final elongation at 72° C. for 7 minutes. The heat block then went to a 10° C. soak cycle.

SPT_AaBG-prom F40:
(SEQ ID NO: 77)
5′-AGCTTAAAGTATGTCCCTTGTCGATGCGATGTATGAATTCATG
AAGCTCAGTTGGCTCGA-3′
SPT_AaBG R40:
(SEQ ID NO: 78)
5′-GATTGATTGTCTACCGCCAGGTGTCAGTCACCTCTAGTTACTA
TTGGACCTTGGGCAGAG-3′

Bold letters represent coding sequence. The remaining sequence is homologous to the region surrounding the insertion sites in the pBM120 vector.

The reaction products were isolated by 0.7% agarose gel electrophoresis using TBE buffer where a 2.66 kb product band was observed. The PCR mixture was digested with Dpn I and purified using a NUCLEOSPIN® TriPrep Kit.

Plasmid pBM120 was digested with Nco I and Pac I, isolated by 0.7% agarose gel electrophoresis using TBE buffer, and purified using a NUCLEOSPIN® TriPrep Kit. The gene fragment and the digested vector were assembled using the yeast extract DNA assembly method described in Example 2 with the S. cerevisiae strain JG169 extract. One μl of the reaction was transformed into E. coli STELLAR™ competent cells according to Example 3. Plasmid DNA was prepared from the E. coli transformants using a BIOROBOT® Universal System. Transformants were confirmed by Bam HI and Nco I digestion and/or DNA sequencing. DNA sequencing of the insertion sites was performed with a Perkin-Elmer Applied Biosystems Model 377 XL Automated DNA Sequencer using dye-terminator chemistry (Giesecke et al., 1992, supra). The primers shown in Table 9 were used for sequencing.

TABLE 9
Sequencing Primers

| Construct | Primer name | Primer sequence (5′→3′) |
|---|---|---|
| pSPT005 | pAllo2F | TGTCCCTTGTCGATGCG |
| | | (SEQ ID NO: 79) |
| | 996271 | ACTCAATTTACCTCTATCCACAC |
| | | TT (SEQ ID NO: 80) |
| | AaBG- | CGGAGATGTTGAAACCATAT |
| | CO_Rev2 | (SEQ ID NO: 81) |

### Example 8: Construction of Plasmid pSPT006

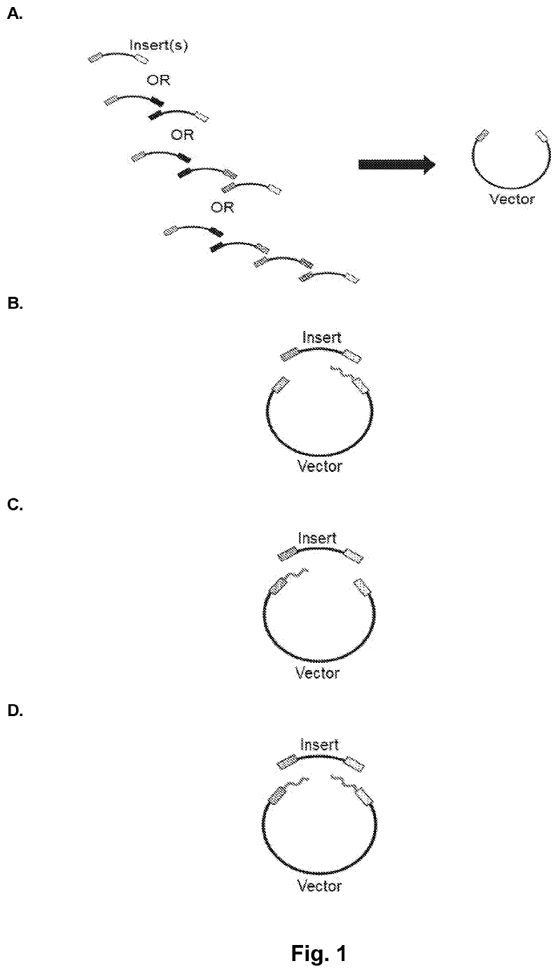

Plasmid pSPT006 was constructed by cloning a codon optimized coding sequence of an Aspergillus aculeatus beta-glucosidase (SEQ ID NO: 1 for the DNA sequence and SEQ ID NO: 2 for the deduced amino acid sequence) into expression vector pBM120 while removing 198 bp and 196 bp of unwanted non-homologous sequences directly upstream and downstream, respectively, to the insertion sites in pBM120 (FIG. 1D).

Primer pairs (shown below) were designed to amplify by PCR the Aspergillus aculeatus beta-glucosidase codon optimized gene from plasmid pDFng181-25 (Example 3). Fifty picomoles of each of the primers above were used in a PCR composed of 10 ng of plasmid pDFng181-15, 1× EXPAND® High Fidelity PCR buffer with MgCl₂, 0.25 mM each of dATP, dTTP, dGTP, and dCTP, and 2.6 units of EXPAND® Enzyme Mix in a final volume of 50 μl. The PCR with the 20 to 30 bp overhangs was performed using a thermal cycler programmed for 1 cycle at 94° C. for 2 minutes; 30 cycles each at 94° C. for 20 seconds, 60° C. for 30 seconds, and 72° C. for 3 minutes; and a final elongation at 72° C. for 7 minutes. The heat block then went to a 10° C. soak cycle.

SPT_AaBG-200prom F40:
(SEQ ID NO: 82)
5′-TCAACCACAAATCACAGTCGTCCCCGGTAATTTAACGGCTATG
AAGCTCAGTTGGCTCGA-3′
SPT_AaBG-term R40:
(SEQ ID NO: 83)
5′-CAGGGACTGTCTGTCTGGTCTTCTACACGAAGGAAAGAGCCTA
TTGGACCTTGGGCAGAG-3′

Bold letters represent coding sequence. The remaining sequence is homologous to the region surrounding the insertion sites of pBM120.

The reaction products were isolated by 0.7% agarose gel electrophoresis using TBE buffer where a 2.66 kb product band was observed. The PCR mixture was digested with Dpn I and purified using a NUCLEOSPIN® TriPrep Kit.

Plasmid pBM120 was digested with Nco I and Pac I, isolated by 0.7% agarose gel electrophoresis using TBE buffer, and purified using a NUCLEOSPin® TriPrep Kit. The gene fragment and the digested vector were assembled using the yeast extract DNA assembly method described in Example 2 with the S. cerevisiae strain JG169 extract. One μl of the reaction was transformed into E. coli STELLAR™ competent cells according to Example 3. Plasmid DNA was prepared from the E. coli transformants using a BIOROBOT® Universal System. Transformants were confirmed by Bam HI and Nco I digestion and/or DNA sequencing. DNA sequencing of the insertion sites was performed with a Perkin-Elmer Applied Biosystems Model 377 XL Automated DNA Sequencer using dye-terminator chemistry (Giesecke et al., 1992, supra). The primers shown in Table 10 were used for sequencing.

TABLE 10
Sequencing Primers

| Construct | Primer name | Primer sequence (5′→3′) |
|---|---|---|
| pSPT006 | pAllo2F | TGTCCCTTGTCGATGCG |
| | | (SEQ ID NO: 84) |
| | pAllo2R | CACATGACTTGGCTTCC |
| | | (SEQ ID NO: 85) |
| | AaBGseq3F | TGGGCCGGCTTGCCCGGTCA |
| | | (SEQ ID NO: 86) |
| | 996271 | ACTCAATTTACCTCTATCCACAC |
| | | TT (SEQ ID NO: 87) |
| | AfBGseq4F | CATTTATGAGTTTGGCCATG |
| | | (SEQ ID NO: 88) |
| | AaBG- | CGGAGATGTTGAAACCATAT |
| | CO_Rev2 | (SEQ ID NO: 89) |

### Example 9: Overall Results of Yeast Extract-Assisted DNA Assembly

The overall results of the Examples demonstrated that the yeast extract assisted DNA assembly method is an efficient and novel restriction site independent cloning method utilizing yeast extract in vitro.

While most of the experiments evaluated the validity of using S. cerevisiae strain JG169 yeast extract to assemble DNA, yeast extract from the S. cerevisiae strain HiP19 was also tested in 2 cases. A simple cloning of 1 insert and 1 digested vector was performed to generate pSPT001 and pSPT002 under the same conditions as Examples 3 and 4, respectively. The HiP19 yeast extract experiments were performed in addition to the JG169 yeast extract experiments to compare the yeast extracts from different strains. This was done to ensure the yeast extract assisted DNA assembly was not dependent solely on S. cerevisiae strain JG169. Both strains were able to produce the desired constructs (Tables 12 and 14).

A range of fragment lengths and vector lengths was also tested to determine if there were any sizing restrictions to this system. Vector lengths from 5.6 kb to 8.7 kb (Table 11) and insert lengths from 395 bp to 2.6 kb (Examples 3-8) were assembled in a variety of combinations. All experiments were successful in producing the desired constructs (Tables 5-7). Four different genes were used to prepare fragments along with 4 different vectors and all worked in yeast extract-assisted in vitro assembly.

TABLE 11

| Vector Name | Vector length |
|---|---|
| pBM120 | 6.8 kb |
| pAllo2 | 5.6 kb |
| pMJ09 | 7.2 kb |

To initially determine the capabilities of the yeast extract assisted DNA assembly, simple assemblies of DNA fragments were tested. An assembly consisting of one vector and one insert was tried with homologous overhangs ranging from 20 bp to 40 bp. Assembly was achieved with as little as 20 bp of overhang with a success rate of 46-83% (Table 12, experiment #1-7). A 3-way assembly was tested next consisting of 2 inserts and 1 vector (Table 12, experiment #8-9). Homologous overhangs with 30 bp were used and were able to generate the anticipated constructs with a success rate of 46-53%. A 4-way assembly was also tested consisting of 3 inserts and 1 vector (Table 12, experiment #10-13). Homologous overhangs with 30-50 bp were used and all were able to produce the correct constructs with a success rate of 16-25%. A 5-way assemble was tested consisting of 4 inserts and 1 vector (Table 12, experiment #14-16). Homologous overhangs with 30-40 bp were used and all were able to produce the correct constructs with a success rate of 6-19%. It was generally observed that as the overhang lengths increased so did the percent of correct clones.

The ability for yeast extract to remove unwanted non-homologous sequence(s) surrounding the insertion sites while simultaneously assembling DNA fragments was also examined. Plasmid pSPT004 was constructed by removing 196 bp of non-homologous sequence directly downstream of the insertion site with a success rate of 38% (Table 13, experiment #17; FIG. 1B). Success rate is the number of correct clones (verified by restriction digestion and/or sequencing)/total number of clones picked. Plasmid pSPT005 was constructed by removing 614 bp of non-homologous sequence directly upstream of the insertion site with a success rate of 69% (Table 13, experiment #18; FIG. 1C). Plasmid pSPT006 was created by removing 198 bp and 196 bp of heterologous sequences directly upstream and downstream, respectively, of the insert (Table 13, experiment #19; FIG. 1D) with a success rate of 38%. The homologous overhangs for pSPT004, pSPT005, and pSPT006 were 40 bp. A trend of increased overhang lengths with increased percent of correct clones was also observed.

The method of the present invention using yeast extract to assemble DNA fragments in vitro is straightforward, economical, and versatile. The yeast extract is simple to make and can be derived from any common laboratory S. cerevisiae strain. The yeast extract can be stored at −80° C. for at least a year and undergo several freeze/thaw cycles with minimal effect to the yield. The method of assembling DNA fragments not only addresses simple cloning with one insert and one vector but also addresses more complicated cloning. The method has been tested for multi-fragment cloning up to 5 fragments or 4 inserts and 1 vector and also has the ability to remove undesired non-homologous sequence(s) at the 5′ and/or 3′ end(s) allowing for restriction site independent cloning and negating the need for specific homologous regions around the insertion sites for DNA recombination.

TABLE 12
S. cerevisiae JG169 Strain Yeast Extract

| | | | | Average | Total | | |
|---|---|---|---|---|---|---|---|
| | | Experiment | Homologous | colonies per | colonies | Total | % |
| | Construct | # | overhangs (bp) | reaction | screened | correct | Correct |
| 2-way (1 insert + | pSPT001 | 1 | 20 | 185 | 40 | 27 | 68 |
| 1 vector) | | 2 | 30 | 96 | 8 | 5 | 63 |
| | | 3 | 40 | 87 | 8 | 6 | 75 |
| | pSPT002 | 4 | 20 | 152 | 48 | 22 | 46 |
| | | 5 | 30 | 250 | 8 | 5 | 63 |
| | | 6 | 40 | 390 | 8 | 5 | 63 |
| | pSPT003 | 7 | 20 | 130 | 24 | 20 | 83 |
| 3-way (2 inserts + | pSPT001 | 8 | 30 | 34 | 32 | 17 | 53 |
| 1 vector) | pSPT002 | 9 | 30 | 63 | 24 | 11 | 46 |
| 4-way (3 inserts + | pSPT001 | 10 | 30 | 45 | 32 | 5 | 16 |
| 1 vector) | | 11 | 40 | 28 | 16 | 3 | 19 |
| | | 12 | 50 | 16 | 24 | 6 | 25 |
| | pSPT002 | 13 | 30 | 70 | 24 | 6 | 25 |
| 5-way (4 inserts + | pSPT001 | 14 | 30 | 82 | 16 | 1 | 6 |
| 1 vector) | | 15 | 40 | 18 | 24 | 4 | 17 |
| | pSPT002 | 16 | 30 | 28 | 16 | 3 | 19 |

TABLE 13
S. cerevisiae JG169 Strain Yeast Extract

| | | Length of | | | | | | |
|---|---|---|---|---|---|---|---|---|
| | | non-homologous | | Homologous | Average | Total | | |
| | | sequence | | overhangs | colonies per | colonies | Total | % |
| | Construct | removed | # | (bp) | reaction | screened | correct | Correct |
| Heterologous | pSPT004 | 196 bp one side | 17 | 40 | 107 | 16 | 6 | 38 |
| sequence | pSPT005 | 614 bp one side | 18 | 40 | 69 | 16 | 11 | 69 |
| removal | pSPT006 | 200 bp both side | 19 | 40 | 32 | 16 | 6 | 38 |

Conditions and results of experiments in the construction of pSPT001-006 using yeast extract from S. cerevisiae strain JG169.

TABLE 14
S. cerevisiae HiP19 Strain Yeast Extract

| | | | Homologous | Average | Total | | |
|---|---|---|---|---|---|---|---|
| | | | overhangs | colonies/ | colonies | Total | % |
| | Construct | # | (bp) | reaction | screened | correct | Correct |
| 2-way (1 insert + | pSPT001 | 21 | 20 | 134 | 32 | 25 | 78 |
| 1 vector) | pSPT002 | 22 | 20 | 211 | 8 | 2 | 25 |

Conditions and results of experiments in the construction of pSPT001-006 using yeast extract from S. cerevisiae strain HiP19.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties.

1. A method of combining a plurality of double-stranded DNA fragments into DNA molecules, said method comprising: contacting the plurality of double-stranded DNA fragments with a cell-free extract of a yeast strain in a single in vitro reaction to combine the plurality of DNA fragments into the DNA molecules, wherein each of the DNA fragments has a 5′ end and a 3′ end, and wherein the DNA fragments assemble with each other when the 5′ end of one fragment has at least 15 bp that are homologous with the 3′ end of another fragment.

2. The method of claim 1, wherein the DNA fragments are obtained by digesting chromosomal DNA with one or more restriction enzymes; by PCR amplifying DNA; by chemical synthesis; or a combination thereof.

3. The method of claim 1, wherein the DNA fragments are obtained from a single genome, two or more genomes, mutated DNA, or shuffled DNA.

4. The method of claim 1, wherein the origin of the DNA fragments is genomic DNA, cDNA, semisynthetic DNA, synthetic DNA, or any combinations thereof.

5. The method of claim 1, wherein the DNA fragments are combined with a linearized plasmid or vector to create a DNA library.

6. The method of claim 5, wherein the DNA library is a mutant DNA library.

7. The method of claim 1, wherein the yeast strain is a Saccharomyces strain.

8. The method of claim 7, wherein the Saccharomyces strain is Saccharomyces cerevisiae.

9. The method of claim 1, wherein the DNA fragments combine with each other when the 5′ end of one fragment has at least 15 bp, at least 20 bp, at least 30 bp, at least 40 bp, at least 50 bp, at least 100 bp, or at least 200 bp that are homologous with the 3′ end of another fragment.

10. The method of claim 1, wherein the in vitro reaction is performed without addition of one or more exogenous enzymes selected from the group consisting of an exogenous DNA restriction enzyme, an exogenous DNA modifying enzyme, an exogenous DNA ligase, and an exogenous DNA polymerase.

11. The method of claim 1, wherein non-homologous sequences of up to at least 1000 bp flanking the homologous region are removed prior to assembling the DNA fragments into DNA molecules.

12. The method of claim 1, further comprising transforming the resulting DNA molecules into a host cell and isolating single colony transformants comprising the DNA molecules.

13. The method of claim 12, wherein the host cell is an E. coli strain.

14. The method of claim 12, further comprising recovering a DNA molecule from the single colony transformants.

15. The method of claim 14, further comprising transforming the recovered DNA molecule into a host cell and selecting a transformant.

16. The method of claim 15, wherein the recovered DNA molecule is operably linked to one or more control sequences that direct the production of a polypeptide having a biological activity of interest encoded by the DNA molecule.

17. The method of claim 15, further comprising cultivating the transformant under conditions suitable for producing the polypeptide having a biological activity of interest.

18. The method of claim 17, further comprising recovering the polypeptide having a biological activity of interest.


SEQUENCE LISTING (89 sequences)

SEQ ID NO: 1 - DNA, 2583 bp, Artificial Sequence
  Artificial DNA Sequence
atgaagctcagttggctcgaggccgccgctctgacggccgcttctgtagtcagcgccgatgaattggctttctctccaccattctacccttcgccttgggctaatggccagggagagtgggcagaagcctaccagcgcgccgtcgccatcgtttctcagatgacactggatgagaaggttaaccttacaacgggtactggatgggaactggagaagtgcgtcggtcaaaccggcggtgttcccagacttaacatcggtggtatgtgtcttcaggattcccctttgggtatccgtgattctgactacaactccgccttccctgctggtgtcaatgtcgccgcgacatgggacaagaacctcgcctaccttcgtggccaggccatgggtcaagaattcagtgacaagggcattgacgttcaactggggcctgctgctggtcctctcggcaggagcccggacggcggcagaaactgggaaggcttctctcctgatccggctctcactggtgtacttttcgccgaaactatcaagggtatccaagacgcgggtgtggtggctactgccaagcattacattctgaatgaacaggagcatttccgacaggttgcagaggccgccggatatggtttcaacatctccgacacgatcagctccaacgtggatgacaagaccattcacgagatgtacctttggccctttgcagatgctgtgcgcgctggcgttggcgctatcatgtgttcctacaatcaaatcaacaacagctacggttgtcaaaacagttacactctcaacaagctcctcaaggctgagctgggcttccaaggcttcgtcatgagtgactggggtgctcaccacagcggtgtcggctctgccctcgctgggttggatatgtcgatgcctggagacattaccttcgactctgccacttccttctggggcacgaacctaactattgctgttcttaacggcaccgttccacagtggcgtgtcgatgacatggctgttcgtatcatggctgcgtactacaaggttggtcgtgaccgtctttaccagccccctaacttcagctcctggacccgggatgagtacggcttcaagtatttctacccccaggagggaccctatgagaaggtgaaccacttcgtcaatgtgcagcgcaaccactctgaggttatccgtaagttgggtgccgacagtacagtgctcttgaagaacaacaatgctcttcctttgaccggcaaggagcgaaaagtggctatcctcggtgaagacgctggttccaactcgtacggtgctaacggctgctctgaccgcggctgtgataacggcactcttgctatggcctggggtagtggtactgccgaattcccttaccttgtcacccccgagcaggctatccaggccgaggtcctcaagcataagggcagcgtctacgctatcactgataactgggctctcagccaggtggagaccctcgcaaaacaagccagcgtgtctttggtgtttgtcaactcggacgcgggagagggttatatcagtgtcgacggcaacgagggtgaccgcaacaatctcactctgtggaagaacggcgacaacctcattaaggctgctgcaaacaactgcaacaacacgattgtggttattcacagtgttgggcccgtcttggttgacgagtggtatgatcaccccaacgtcactgccatcctctgggccggcttgcccggtcaggagagtggcaactccctggctgacgtgctctatggccgcgtcaaccccggcgccaagtctccgttcacctggggcaagactcgggaggcttacggggattacttggtccgtgagctcaacaatggcaatggtgctccccaggatgatttctcggagggcgtcttcattgactaccgtggatttgacaagcgcaatgagacccccatttatgagtttggccatggcttgagctacaccacctttaactactctggccttcacatccaggttctcaatgcttcgagtaacgcacaagtcgccactgagaccggcgccgcgccaaccttcggtcaagtcggtaatgccagtgactacgtgtatcccgagggtctcaccagaattagcaagtttatttacccttggctcaactcgaccgacctcaaggcctcttctggcgacccgtactacggcgtcgacaccgctgagcacgtgcccgaaggcgctactgatgggtctcctcaacccgttctgcctgctggcggcggcttcggtggtaaccctagactttatgatgagcttatcagggtgtcggtgaccgtcaagaacactggtcgtgtcgccggtgatgctgtccctcaattgtatgtttcactgggcggaccgaacgagcctaaggtcgttctgcgcaagttcgaccgactcaccctgaagccttccgaggagacggtttggaccacgactcttacccgtcgtgatctctctaattgggatgtggccgctcaggactgggtcatcacatcttaccccaagaaagtgcacgtcggcagctcctcgcgtcagctgcctctgcacgcggctctgcccaaggtccaatag

SEQ ID NO: 2 - protein, 860 aa, Aspergillus aculeatus
MKLSWLEAAALTAASVVSADELAFSPPFYPSPWANGQGEWAEAYQRAVAIVSQMTLDEKVNLTTGTGWELEKCVGQTGGVPRLNIGGMCLQDSPLGIRDSDYNSAFPAGVNVAATWDKNLAYLRGQAMGQEFSDKGIDVQLGPAAGPLGRSPDGGRNWEGFSPDPALTGVLFAETIKGIQDAGVVATAKHYILNEQEHFRQVAEAAGYGFNISDTISSNVDDKTIHEMYLWPFADAVRAGVGAIMCSYNQINNSYGCQNSYTLNKLLKAELGFQGFVMSDWGAHHSGVGSALAGLDMSMPGDITFDSATSFWGTNLTIAVLNGTVPQWRVDDMAVRIMAAYYKVGRDRLYQPPNFSSWTRDEYGFKYFYPQEGPYEKVNHFVNVQRNHSEVIRKLGADSTVLLKNNNALPLTGKERKVAILGEDAGSNSYGANGCSDRGCDNGTLAMAWGSGTAEFPYLVTPEQAIQAEVLKHKGSVYAITDNWALSQVETLAKQASVSLVFVNSDAGEGYISVDGNEGDRNNLTLWKNGDNLIKAAANNCNNTIVVIHSVGPVLVDEWYDHPNVTAILWAGLPGQESGNSLADVLYGRVNPGAKSPFTWGKTREAYGDYLVRELNNGNGAPQDDFSEGVFIDYRGFDKRNETPIYEFGHGLSYTTFNYSGLHIQVLNASSNAQVATETGAAPTFGQVGNASDYVYPEGLTRISKFIYPWLNSTDLKASSGDPYYGVDTAEHVPEGATDGSPQPVLPAGGGFGGNPRLYDELIRVSVTVKNTGRVAGDAVPQLYVSLGGPNEPKVVLRKFDRLTLKPSEETVWTTTLTRRDLSNWDVAAQDWVITSYPKKVHVGSSSRQLPLHAALPKVQ

SEQ ID NO: 3 - DNA, 40 bp, Artificial Sequence
  Artificial DNA Primer
ctctatatacacaactggccatgaagctcagttggctcga

SEQ ID NO: 4 - DNA, 40 bp, Artificial Sequence
  Artificial DNA Primer
gtgtcagtcacctctagttactattggaccttgggcagag

SEQ ID NO: 5 - DNA, 50 bp, Artificial Sequence
  Artificial DNA Primer
ttcctcaatcctctatatacacaactggccatgaagctcagttggctcga

SEQ ID NO: 6 - DNA, 50 bp, Artificial Sequence
  Artificial DNA Primer
ctaccgccaggtgtcagtcacctctagttactattggaccttgggcagag

SEQ ID NO: 7 - DNA, 60 bp, Artificial Sequence
  Artificial DNA Primer
cttctcttccttcctcaatcctctatatacacaactggccatgaagctcagttggctcga

SEQ ID NO: 8 - DNA, 60 bp, Artificial Sequence
  Artificial DNA Primer
gattgattgtctaccgccaggtgtcagtcacctctagttactattggaccttgggcagag

SEQ ID NO: 9 - DNA, 50 bp, Artificial Sequence
  Artificial DNA Primer
gcagccgttagcaccgtacgagttggaaccagcgtcttcaccgaggatag

SEQ ID NO: 10 - DNA, 50 bp, Artificial Sequence
  Artificial DNA Primer
ggttccaactcgtacggtgctaacggctgctctgaccgcggctgtgataa

SEQ ID NO: 11 - DNA, 50 bp, Artificial Sequence
  Artificial DNA Primer
gggcagagccgacaccgctgtggtgagcaccccagtcactcatgacgaag

SEQ ID NO: 12 - DNA, 50 bp, Artificial Sequence
  Artificial DNA Primer
gtgctcaccacagcggtgtcggctctgccctcgctgggttggatatgtcg

SEQ ID NO: 13 - DNA, 50 bp, Artificial Sequence
  Artificial DNA Primer
actctcctgaccgggcaagccggcccagaggatggcagtgacgttggggt

SEQ ID NO: 14 - DNA, 50 bp, Artificial Sequence
  Artificial DNA Primer
ctctgggccggcttgcccggtcaggagagtggcaactccctggctgacgt

SEQ ID NO: 15 - DNA, 50 bp, Artificial Sequence
  Artificial DNA Primer
cacgttggagctgatcgtgtcggagatgttgaaaccatatccggcggcct

SEQ ID NO: 16 - DNA, 50 bp, Artificial Sequence
  Artificial DNA Primer
aacatctccgacacgatcagctccaacgtggatgacaagaccattcacga

SEQ ID NO: 17 - DNA, 50 bp, Artificial Sequence
  Artificial DNA Primer
catggccaaactcataaatgggggtctcattgcgcttgtcaaatccacgg

SEQ ID NO: 18 - DNA, 50 bp, Artificial Sequence
  Artificial DNA Primer
atgagacccccatttatgagtttggccatggcttgagctacaccaccttt

SEQ ID NO: 19 - DNA, 60 bp, Artificial Sequence
  Artificial DNA Primer
tcagagcagccgttagcaccgtacgagttggaaccagcgtcttcaccgaggatagccact

SEQ ID NO: 20 - DNA, 60 bp, Artificial Sequence
  Artificial DNA Primer
acgctggttccaactcgtacggtgctaacggctgctctgaccgcggctgtgataacggca

SEQ ID NO: 21 - DNA, 60 bp, Artificial Sequence
  Artificial DNA Primer
agcgagggcagagccgacaccgctgtggtgagcaccccagtcactcatgacgaagccttg

SEQ ID NO: 22 - DNA, 60 bp, Artificial Sequence
  Artificial DNA Primer
ctggggtgctcaccacagcggtgtcggctctgccctcgctgggttggatatgtcgatgcc

SEQ ID NO: 23 - DNA, 60 bp, Artificial Sequence
  Artificial DNA Primer
ttgccactctcctgaccgggcaagccggcccagaggatggcagtgacgttggggtgatca

SEQ ID NO: 24 - DNA, 60 bp, Artificial Sequence
  .Artificial DNA Primer
ccatcctctgggccggcttgcccggtcaggagagtggcaactccctggctgacgtgctct

SEQ ID NO: 25 - DNA, 60 bp, Artificial Sequence
  Artificial DNA Primer
tcatccacgttggagctgatcgtgtcggagatgttgaaaccatatccggcggcctctgca

SEQ ID NO: 26 - DNA, 60 bp, Artificial Sequence
  Artificial DNA Primer
gtttcaacatctccgacacgatcagctccaacgtggatgacaagaccattcacgagatgt

SEQ ID NO: 27 - DNA, 60 bp, Artificial Sequence
  Artificial DNA Primer
caagccatggccaaactcataaatgggggtctcattgcgcttgtcaaatccacggtagtc

SEQ ID NO: 28 - DNA, 60 bp, Artificial Sequence
  Artificial DNA Primer
gcgcaatgagacccccatttatgagtttggccatggcttgagctacaccacctttaacta

SEQ ID NO: 29 - DNA, 70 bp, Artificial Sequence
  Artificial DNA Primer
tctatccacacttctcttccttcctcaatcctctatatacacaactggccatgaagctcagttggctcga

SEQ ID NO: 30 - DNA, 70 bp, Artificial Sequence
  Artificial DNA Primer
tagcgaaatggattgattgtctaccgccaggtgtcagtcacctctagttactattggaccttgggcagag

SEQ ID NO: 31 - DNA, 70 bp, Artificial Sequence
  Artificial DNA Primer
cgcggtcagagcagccgttagcaccgtacgagttggaaccagcgtcttcaccgaggatagccacttttcg

SEQ ID NO: 32 - DNA, 70 bp, Artificial Sequence
  Artificial DNA Primer
tgaagacgctggttccaactcgtacggtgctaacggctgctctgaccgcggctgtgataacggcactctt

SEQ ID NO: 33 - DNA, 70 bp, Artificial Sequence
  Artificial DNA Primer
aacccagcgagggcagagccgacaccgctgtggtgagcaccccagtcactcatgacgaagccttggaagc

SEQ ID NO: 34 - DNA, 70 bp, Artificial Sequence
  Artificial DNA Primer
agtgactggggtgctcaccacagcggtgtcggctctgccctcgctgggttggatatgtcgatgcctggag

SEQ ID NO: 35 - DNA, 70 bp, Artificial Sequence
  Artificial DNA Primer
gggagttgccactctcctgaccgggcaagccggcccagaggatggcagtgacgttggggtgatcatacca

SEQ ID NO: 36 - DNA, 70 bp, Artificial Sequence
  Artificial DNA Primer
cactgccatcctctgggccggcttgcccggtcaggagagtggcaactccctggctgacgtgctctatggc

SEQ ID NO: 37 - DNA, 70 bp, Artificial Sequence
  Artificial DNA Primer
tcttgtcatccacgttggagctgatcgtgtcggagatgttgaaaccatatccggcggcctctgcaacctg

SEQ ID NO: 38 - DNA, 70 bp, Artificial Sequence
  Artificial DNA Primer
atatggtttcaacatctccgacacgatcagctccaacgtggatgacaagaccattcacgagatgtacctt

SEQ ID NO: 39 - DNA, 70 bp, Artificial Sequence
  Artificial DNA Primer
tagctcaagccatggccaaactcataaatgggggtctcattgcgcttgtcaaatccacggtagtcaatga

SEQ ID NO: 40 - DNA, 70 bp, Artificial Sequence
  Artificial DNA Primer
gacaagcgcaatgagacccccatttatgagtttggccatggcttgagctacaccacctttaactactctg

SEQ ID NO: 41 - DNA, 25 bp, Artificial Sequence
  Artificial DNA Primer
actcaatttacctctatccacactt

SEQ ID NO: 42 - DNA, 17 bp, Artificial Sequence
  Artificial DNA Primer
cacatgacttggcttcc

SEQ ID NO: 43 - DNA, 20 bp, Artificial Sequence
  Artificial DNA Primer
gtacttttcgccgaaactat

SEQ ID NO: 44 - DNA, 20 bp, Artificial Sequence
  Artificial DNA Primer
acggcttcaagtatttctac

SEQ ID NO: 45 - DNA, 20 bp, Artificial Sequence
  Artificial DNA Primer
tgggccggcttgcccggtca

SEQ ID NO: 46 - DNA, 1581 bp, Artificial Sequence
  Artificial DNA Sequence
atgcttttgcaagccttccttttccttttggctggttttgcagccaagatctctgcacagcaggccggcaccctgaccaccgaaacccatccttctctgacctggcagcaatgctctgccggcggcagctgcaccactcagaacggcaaggtcgtcatcgacgccaactggcgctgggttcacagcaccagcggctcgaacaactgctacactggcaacacttgggatgctactctctgccctgacgacgtgacttgcgctgccaactgcgccctggacggcgctgactactcgggcacctacggtgtcaccaccagcggcaactctctgcgcctgaacttcgtcacccaggcgtcgcagaagaacgtcggctctcgtctctatctgatggagaatgacacaacctaccagatcttcaagttgctgaaccaggagttcacctttgacgttgatgtctccaaccttccctgcggtctcaacggtgctctctacctggttgccatggatgccgacggcggcatggccaagtacccaaccaacaaggctggtgcgaagtacggaaccggttactgcgactcccagtgccctcgcgacctgaagttcatcaacggtgaggccaatgttgagggatggcagccttcttccaatgaccccaactctggcattggcaaccacggctcttgctgtgctgagatggacatctgggaggccaacagcatctccaatgcagtcactcctcacccttgcgacaccccgggacaggtcatgtgcaccggcaacaactgtggtggcacttacagcactactcgctatgctggcacttgcgatcctgatggctgcgacttcaacccctaccgcatgggcaaccactccttctacggccccaaacagatcgtcgacaccagctccaagttcactgttgttactcagttcctcaccgatgatggcacctccaccggcaccctcagcgagatcaggcgcttctacgttcagaacggccaggtcatccccaactccgtgtccacgatcagcggcgtctccggcaactccatcaccaccgagttctgcacggcccagaagcaggctttcggcgacactgatgacttcagcaagcacggcggtctgtctggcatgtccgccgccctctcccagggtatggttctcgtcatgagcttgtgggacgaccacgccgccaacatgctctggcttgacagcacctacccgaccaacgccacctcttccacccccggtgccgcccgtggtacttgcgacatctcctccggtgtccccgccgatgttgagtccaacgaccccaacgcctacgtcgtctactccaacatcaaggtcggcccgatcggctctaccttcagcagctctggctctggctctagctccagctccagcaccaccaccaccaccaccgcttccccaaccacgaccacctccagcgcttccagcaccggcactggcgttgctcagcactggggtcagtgcggtggccagggatggaccggtccgaccacctgcgttagcccctacacctgccaggagctgaacccctactactaccagtgcctgtaa

SEQ ID NO: 47 - protein, 526 aa, Talaromyces leycettanus
MLLQAFLFLLAGFAAKISAQQAGTLTTETHPSLTWQQCSAGGSCTTQNGKVVIDANWRWVHSTSGSNNCYTGNTWDATLCPDDVTCAANCALDGADYSGTYGVTTSGNSLRLNFVTQASQKNVGSRLYLMENDTTYQIFKLLNQEFTFDVDVSNLPCGLNGALYLVAMDADGGMAKYPTNKAGAKYGTGYCDSQCPRDLKFINGEANVEGWQPSSNDPNSGIGNHGSCCAEMDIWEANSISNAVTPHPCDTPGQVMCTGNNCGGTYSTTRYAGTCDPDGCDFNPYRMGNHSFYGPKQIVDTSSKFTVVTQFLTDDGTSTGTLSEIRRFYVQNGQVIPNSVSTISGVSGNSITTEFCTAQKQAFGDTDDFSKHGGLSGMSAALSQGMVLVMSLWDDHAANMLWLDSTYPTNATSSTPGAARGTCDISSGVPADVESNDPNAYVVYSNIKVGPIGSTFSSSGSGSSSSSSTTTTTTASPTTTTSSASSTGTGVAQHWGQCGGQGWTGPTTCVSPYTCQELNPYYYQCL

SEQ ID NO: 48 - DNA, 40 bp, Artificial Sequence
  Artificial DNA Primer
atatacacaactggatttacatgcttttgcaagccttcct

SEQ ID NO: 49 - DNA, 40 bp, Artificial Sequence
  Artificial DNA Primer
gtgtcagtcacctctagttattacaggcactggtagtagt

SEQ ID NO: 50 - DNA, 50 bp, Artificial Sequence
  Artificial DNA Primer
tcaatcctctatatacacaactggatttacatgcttttgcaagccttcct

SEQ ID NO: 51 - DNA, 50 bp, Artificial Sequence
  Artificial DNA Primer
ctaccgccaggtgtcagtcacctctagttattacaggcactggtagtagt

SEQ ID NO: 52 - DNA, 60 bp, Artificial Sequence
  Artificial DNA Primer
tcttccttcctcaatcctctatatacacaactggatttacatgcttttgcaagccttcct

SEQ ID NO: 53 - DNA, 60 bp, Artificial Sequence
  Artificial DNA Primer
gattgattgtctaccgccaggtgtcagtcacctctagttattacaggcactggtagtagt

SEQ ID NO: 54 - DNA, 50 bp, Artificial Sequence
  Artificial DNA Primer
gtagtgctgtaagtgccaccacagttgttgccggtgcacatgacctgtcc

SEQ ID NO: 55 - DNA, 50 bp, Artificial Sequence
  Artificial DNA Primer
caacaactgtggtggcacttacagcactactcgctatgctggcacttgcg

SEQ ID NO: 56 - DNA, 50 bp, Artificial Sequence
  Artificial DNA Primer
ttgttggttgggtacttggccatgccgccgtcggcatccatggcaaccag

SEQ ID NO: 57 - DNA, 50 bp, Artificial Sequence
  Artificial DNA Primer
cggcggcatggccaagtacccaaccaacaaggctggtgcgaagtacggaa

SEQ ID NO: 58 - DNA, 50 bp, Artificial Sequence
  Artificial DNA Primer
tgcagaactcggtggtgatggagttgccggagacgccgctgatcgtggac

SEQ ID NO: 59 - DNA, 50 bp, Artificial Sequence
  Artificial DNA Primer
ccggcaactccatcaccaccgagttctgcacggcccagaagcaggctttc

SEQ ID NO: 60 - DNA, 50 bp, Artificial Sequence
  Artificial DNA Primer
tggtaggttgtgtcattctccatcagatagagacgagagccgacgttctt

SEQ ID NO: 61 - DNA, 50 bp, Artificial Sequence
  Artificial DNA Primer
ctatctgatggagaatgacacaacctaccagatcttcaagttgctgaacc

SEQ ID NO: 62 - DNA, 50 bp, Artificial Sequence
  Artificial DNA Primer
gcatgttggcggcgtggtcgtcccacaagctcatgacgagaaccataccc

SEQ ID NO: 63 - DNA, 50 bp, Artificial Sequence
  Artificial DNA Primer
gcttgtgggacgaccacgccgccaacatgctctggcttgacagcacctac

SEQ ID NO: 64 - DNA, 17 bp, Artificial Sequence
  Artificial DNA Primer
tgtcccttgtcgatgcg

SEQ ID NO: 65 - DNA, 17 bp, Artificial Sequence
  Artificial DNA Primer
cacatgacttggcttcc

SEQ ID NO: 66 - DNA, 1143 bp, Artificial Sequence
  Artificial DNA Sequence
atgaagttccatctggcatctgtggccgccgggctgtcggcattgatcaccgccgtctacggccagtgcgatctcccatcgagctaccgctggtcctcgagcggtgtactcaccgatcccaagaacggctgggcctccctcaaggacttcaccattgctccctacaacggccagcacctggtgtatgccaccaaggttgagaatggcaactggggctccatggccttcggtctcttcagcgacttcagcagcatgagctccgtcgcccagaacggcatgagctcgtccaccgtcgctcccacgctcttctacttcgcccccaagagcgtctggatcctggcctaccaatggggcgcctggccgttcatctacaggacgtccaccaaccccaccaaccagaacggctggtctgcgccccaggccctgttcaccggatccatcaccggatcgtcgaccggcccgatcgaccagacgctgatcggcgacggcaccaacatgtacctcttcttcaccggcgacaacggcaagatctaccgtgccagcatgcccatcggcaacttcccgggcagcttcggctccaactacgtcaccgtcctgagcgacaccaccaacaacctcttcgaagccgtccaggtgtacaagctcaagggcctccagaagtacctcatgatcgtcgaggccatcggcgccaacggccgctacttccgctccttcaccgctaccagccttggtggttcctggacccttcaggccggttccgagagcagcccgttcgccggcaaggccaacagcggcgccacctggaccaacgacatcagccacggcgagctcgtccgctcctcgagcgaccagaccttcgaggtcgacgcctgcaacctgcgcctgctgtaccagggccgcaacccgagctccggcggcgactacacccttctgccgtaccgcccgggtctgctgacgctgcagaaccccagcggcaacccgcagcccgtgactacgacgacgcggaccaccagctcgcctcagccgacgggggcttgtgcgccgctttacggtcagtgtggtggaagtgggtggactgggcctacgtgctgcgcgagcggtacctgcaaggccgggaacgagtggtactctcagtgcacttag

SEQ ID NO: 67 - protein, 380 aa, Gliomastix murorum
MKFHLASVAAGLSALITAVYGQCDLPSSYRWSSSGVLTDPKNGWASLKDFTIAPYNGQHLVYATKVENGNWGSMAFGLFSDFSSMSSVAQNGMSSSTVAPTLFYFAPKSVWILAYQWGAWPFIYRTSTNPTNQNGWSAPQALFTGSITGSSTGPIDQTLIGDGTNMYLFFTGDNGKIYRASMPIGNFPGSFGSNYVTVLSDTTNNLFEAVQVYKLKGLQKYLMIVEAIGANGRYFRSFTATSLGGSWTLQAGSESSPFAGKANSGATWTNDISHGELVRSSSDQTFEVDACNLRLLYQGRNPSSGGDYTLLPYRPGLLTLQNPSGNPQPVTTTTRTTSSPQPTGACAPLYGQCGGSGWTGPTCCASGTCKAGNEWYSQCT

SEQ ID NO: 68 - DNA, 40 bp, Artificial Sequence
  Artificial DNA Primer
gtcaaccgcggactgcgcacatgaagttccatctggcatc

SEQ ID NO: 69 - DNA, 40 bp, Artificial Sequence
  Artificial DNA Primer
ggctttcgccacggagcttactaagtgcactgagagtacc

SEQ ID NO: 70 - DNA, 20 bp, Artificial Sequence
  Artificial DNA Primer
cccatctactcatcaactca

SEQ ID NO: 71 - DNA, 20 bp, Artificial Sequence
  Artificial DNA Primer
ataaatcacccggggccatg

SEQ ID NO: 72 - DNA, 60 bp, Artificial Sequence; identical to SEQ ID NO: 7
  Artificial DNA Primer

SEQ ID NO: 73 - DNA, 60 bp, Artificial Sequence
  Artificial DNA Primer
cagggactgtctgtctggtcttctacacgaaggaaagagcctattggaccttgggcagag

SEQ ID NO: 74 - DNA, 17 bp, Artificial Sequence
  Artificial DNA Primer
cacatgacttggcttcc

SEQ ID NO: 75 - DNA, 20 bp, Artificial Sequence
  Artificial DNA Primer
tgggccggcttgcccggtca

SEQ ID NO: 76 - DNA, 20 bp, Artificial Sequence
  Artificial DNA Primer
catttatgagtttggccatg

SEQ ID NO: 77 - DNA, 60 bp, Artificial Sequence
  Artificial DNA Primer
agcttaaagtatgtcccttgtcgatgcgatgtatgaattcatgaagctcagttggctcga

SEQ ID NO: 78 - DNA, 60 bp, Artificial Sequence; identical to SEQ ID NO: 8
  Artificial DNA Primer

SEQ ID NO: 79 - DNA, 17 bp, Artificial Sequence
  Artificial DNA Primer
tgtcccttgtcgatgcg

SEQ ID NO: 80 - DNA, 25 bp, Artificial Sequence
  Artificial DNA Primer
actcaatttacctctatccacactt

SEQ ID NO: 81 - DNA, 20 bp, Artificial Sequence
  Artificial DNA Primer
cggagatgttgaaaccatat

SEQ ID NO: 82 - DNA, 60 bp, Artificial Sequence
  Artificial DNA Primer
tcaaccacaaatcacagtcgtccccggtaatttaacggctatgaagctcagttggctcga

SEQ ID NO: 83 - DNA, 60 bp, Artificial Sequence; identical to SEQ ID NO: 73
  Artificial DNA Primer

SEQ ID NO: 84 - DNA, 17 bp, Artificial Sequence
  Artificial DNA Primer
tgtcccttgtcgatgcg

SEQ ID NO: 85 - DNA, 17 bp, Artificial Sequence
  Artificial DNA Primer
cacatgacttggcttcc

SEQ ID NO: 86 - DNA, 20 bp, Artificial Sequence
  Artificial DNA Primer
tgggccggcttgcccggtca

SEQ ID NO: 87 - DNA, 25 bp, Artificial Sequence
  Artificial DNA Primer
actcaatttacctctatccacactt

SEQ ID NO: 88 - DNA, 20 bp, Artificial Sequence
  Artificial DNA Primer
catttatgagtttggccatg

SEQ ID NO: 89 - DNA, 20 bp, Artificial Sequence
  Artificial DNA Primer
cggagatgttgaaaccatat